(19) United States
(12) Patent Application Publication
(10) Pub. No.: US 2011/0321180 A1
(43) Pub. Date: Dec. 29, 2011

(54) COMPOSITIONS AND METHODS TO GENERATE PILOSEBACEOUS UNITS

(75) Inventors: Lily Lee, Los Angeles, CA (US); Cheng Ming Chuong, Los Angeles, CA (US); Warren Garner, Los Angeles, CA (US); Ting Xin Jiang, Los Angeles, CA (US)

(73) Assignee: University of Southern California

(21) Appl. No.: 13/130,296

(22) PCT Filed: Nov. 19, 2009
(86) PCT No.: PCT/US09/65202

§ 371 Date: Aug. 8, 2011

Related U.S. Application Data
(60) Provisional application No. 61/116,620, filed on Nov. 20, 2008.

(51) Int. Cl. A01K 67/027; A61K 35/36; A61K 49/00; C12N 5/071

(52) U.S. Cl. 800/9; 435/395; 435/375; 435/377; 424/93.7; 424/9.8

(57) ABSTRACT

The invention provides compositions and methods to generate pilosebaceous units. In one aspect, the invention comprises a biocompatible scaffold and an effective amount of dermal and epidermal precursor cells.

### CROSS-REFERENCE TO RELATED APPLICATIONS

This application claims the benefit under 35 U.S.C. §119(e) of U.S. Provisional Application Ser. No. 61/116,620, filed Nov. 20, 2008, the content of which is incorporated by reference into the present disclosure in its entirety.

### STATEMENT OF FEDERAL SUPPORT

This invention was supported by grants from the National Institutes of Health (Grant Nos. AR 047364 and F32 GM08019). The government has rights in this application.

### BACKGROUND

The skin consists of two layers, an epidermis layer derived from the ectoderm, and a dermis layer derived from mesoderm underneath the epidermis layer. Dermis is subdivided into two strata, the superficial papillary layer and the reticular layer. The epidermis is a stratified squamous epithelium. The epidermis is the outer layer of the skin and acts as a protective film against external insults. The majority of epidermal cells undergo keratinization and form the dead superficial layers of the skin. The thickness of the epidermis is maintained at a constant level by continuous exchange of keratinocytes from the basal layer to the surface where they lose the nucleus, keratinize, die and desquamate. Wounds at the epidermal level heal by epithelialization due to keratinocyte migration and mitosis. When the epidermis is damaged a blood clot is formed, forming a scab that covers and protects the dermis. Cells from the margins of the wounds, undamaged lower layers, dermal sebaceous glands and hair follicles start to migrate to the wounded site and form a new epidermis layer.

Thousands of people suffer traumatic burns every year. While most survive, the deformities and scarring can cause lifelong suffering. Medical care for burn patients has advanced in the last few decades. This is due, in large part, to medical research that has contributed to the management of fluid loss and infection. Now, morbidity rather than mortality is the main issue in burn care. Technology has advanced such that doctors can save lives by replacing damaged or missing skin with skin autografting or dermal equivalent products made from animal products. However, even with the new advances in skin substitutes, grafted skin is unable to completely restore normal skin function. For skin to function normally, it requires multiple specialized organs within that work together for optimal utility. These include hair, sebaceous (oil) and other skin appendages. Sebaceous glands are necessary for normal skin lubrication. Any patient with significant skin lose has a lifelong requirement for daily moisturizer to replace skin oils. Hair functions in direct relationship to external appearance which directly influences a patient's quality of life.

Burns can cause lasting appearance and functional defects to visible areas on the skin. Research has taken today's burn care beyond just keeping the patient alive and fighting against infection and fluid loss. Appearance can profoundly affect one's physical and psychological well-being, especially when altered after a severe disfiguring injury. Current technology has improved the mortality rate of burns tremendously, but improvement of the morbidity rate of burn injuries has a long way to go.

Currently, burn victims have scars, which never look or feel like normal skin. The main difference between scar and normal skin lies in the fact that scar does not have hair, sweat glands, or oil glands. While one may find these to be ancillary, lack of any or all of these causes significant suffering in a patient with scars. Evolutionarily, hair kept people warm. Today, billions of dollars are spent nationwide in the grooming and restoration of hair. It is conceivable that the benefit of the ability to use stem cells to grow hairs can be extended beyond restoration of normal skin architecture.

The current gold standard of treatment for hair loss is hair transplantation, which is a laborious and expensive procedure. Essentially, a strip of hair bearing skin is surgically taken from the patient. The individual hair follicles are then painstakingly dissected out one by one and planted into small slits are made on the recipient's skin. Patients who do not have enough hair as a result of previous injury (such as burn), cannot spare extra hair to transplant. There is just no way to increase the total number of hairs.

A goal of current bioengineering is to generate or reconstitute organized and functional hair follicle-bearing skin. It is known that the hair follicle has profound regenerative capability and it cycles over the lifetime of the individual. The normal cycling of hair through stages of growth, shedding and then regrowth has been used as a model for stem cell based regeneration.

In embryonic development, hair follicles are built stepwise (Millar (2002) Invest. Dermatol. 118:216-25; Fuchs (2007) Nature 445:834-42; Plikus et al. Mouse skin ectodermal organs. In: The Mouse in Biomedical Research (Fox, Barthold, Davisson, Newcomer, Quimby and Smith, eds), 2^{nd} edn, Vol 3, Academic Press: Amsterdam, 2007). During this process, molecular signals interact among tissues and multiple morphogenetic events occur; some are regulated by the mesenchyme and some by the epithelium. If one accepts the fact that hair-follicle development involves many molecular and cellular events embedded in discrete morphogenetic steps, then it would not be so surprising to encounter incomplete and imperfect structures as the science advances to the engineering of hair follicles—it is hard to get every step right in this dawn of bioengineering.

The salient events of hair-follicle morphogenesis can be summarized as follows: formation of dermal condensations→epithelial invagination to form the follicular wall→formation of DP at the base of the follicle→topologic arrangement of localized stem, transient amplifying (TA), and differentiated cell clusters→morphogenesis to build the architecture of different hair types in the differentiating zone→molecular differentiation of hair-shaft components→ability to shed hairs while preserving stem cells and DP for the next cycle→ability to regenerate. Failure of any of these events will lead to disrupted hair-follicle structures, resulting in various degrees of incomplete hair-follicle formation (Chuong et al. (2007) J. Invest. Dermatol. 127(9):2098-100).

It has been shown that it is possible to use dissociated hair precursor cells to produce hair follicles in vivo (Weinberg et al. (1993) J. Invest. Dermatol. 100(3):229-36; Lichit et al. (1993) J. Invest. Dermatol. 101(1 Suppl):124S-129S). However, the procedure is time consuming and cumbersome. The method is only good for laboratory purposes on animal research. Recently, a simplified procedure was produced by injecting the same dissociated precursor cells underneath the skin of mice. While hair follicles and its associated appendages do form with this method, they aggregate in a random fashion as cysts underneath the dermis (Zheng et al. (2005) J. Invest. Dermatol. 124(5):867-76). Nevertheless, even with these shortcomings, this procedure is good for easily assaying molecules on a short term basis and important for studying the biochemical and physiological mechanisms governing hair formation. However, due to the fact that the environment in which the growth of these hairs is trapped, the hair grows on the underside and cannot cycle and thus is not practical for clinical use.

Thus, there is still a need for a simple, reproducible and efficient procedure that can generate a large number of pilosebaceous units with a clinically acceptable appearance.

### SUMMARY OF THE INVENTION

This invention provides a new procedure that allows multipotential skin precursor cells to form a large number of new hair follicles which are arranged in a physiological plane with a cosmetically acceptable appearance. This procedure can be performed efficiently, reproducibly and on a large scale so as to be appropriate for clinical applications.

Thus, in one aspect this invention provides a composition to generate pilosebaceous units in a physiological plane comprising a biocompatible scaffold and an effective amount of skin precursor cells. In one aspect, the skin precursor cells comprise epidermal precursor cells and dermal precursor cells. In another aspect, the composition may further comprise an effective amount of a growth or differentiation factor that promotes the growth and differentiation of dermal and epidermal precursor cells. Examples of such factors include, but are not limited to Bone Morphogenic Protein (BMP) inhibitors such as noggin, chordin, gremlin, dorsomorphin, sclerostin and follistatin and any combination thereof. Examples of such factors may also include, but are not limited to Platelet Derived Growth Factor (PDGF), Vascular Endothelial Growth Factor (VEGF), Epithelial Growth Factor (EGF), TGF-β, Fibroblast Growth Factor (FGF), insulin, transferrin, retinoid, and any combination thereof. In another aspect, the composition further comprises an effective amount of minoxidil, finasteride or an agent enhancing hair growth.

In one aspect of this invention, the composition may be prepared by admixing an effective amount of isolated skin precursor cells in serum-free medium and a biocompatible scaffold, under conditions that favor the incorporation of the cells into the biocompatible scaffold. In some embodiments, the concentration of the cells in the scaffold is from about 800,000 cells/mm³ to about 1,500,000 cells/mm³. In some embodiments, the scaffold and cells are admixed by passively contacting the cells with the scaffold at a temperature range from about 25 to about 37° C. for about 30 minutes to about 2 hours.

Another aspect of this invention provides a method for generating pilosebaceous units in a physiological plane in a mammal in need thereof, comprising implanting the composition of this invention into the dermal layer of the mammal under conditions that favor implantation of the composition into the dermis of the mammal. In some embodiments, the conditions that favor implantation of the composition into the dermis of the mammal comprise suitable pressure to maintain contact between the composition and the muscle or subcutaneous fat of the mammal for at least 3 days.

### BRIEF DESCRIPTION OF THE FIGURES

FIG. 1 shows hair formation with the method described in Zheng et al. J. Invest. Dermatol. (2005) 124(5):867-76. FIG. 1A shows that random arrangement of hairs grow within a hair cyst at fourteen days after injection of the dissociated precursor cells underneath the skin. FIG. 1B shows that with Lentivirus-Green Fluorescent Protein (GFP), lineage of cells was traced to formation of hairs.

FIG. 2 shows hair formation with the method of the present invention. FIG. 2A shows the wound at day seven post graft, after the sterile dressing and silicone protective layer were removed. FIG. 2B shows full and robust hair growth by days 15 to 20.

FIG. 3 shows close up views of the hair formation. Note that hair follicles are in the same physiological plane as proved by direct visualization, tissue sectioning or ultrasound.

FIG. 4 shows that Integra matrix may be shaped to specific size and shape to fit various clinical needs. FIG. 4A shows a single long strip grafted with multipotential cells. Hair growth on this strip simulates a human eyebrow. FIGS. 4B and 4C show that the top of the calvarium may be grafted with Integra matrix carrying hair bearing cells.

FIGS. 5A-L illustrate step by step the hair forming protocol: (A) Mice are cleaned and prepared for surgery under anesthesia. (B-C) The approximate area of skin to be grafted for hair bearing is cut out in a full thickness layer, note musculature. (D) An example of scaffold that has been seeded with cells using a pipet and allowed to dry briefly. Scaffold is sitting on a protective silicone membrane. (E) Placement of scaffold and cells over recipient bed. (F-G) Simple interrupted sutures to secure graft in place. (H-I) Antimicrobial ointment and gauze used to dress the wound. (J-L) Securing of dressing with a tight elastic wrapping allows for better adherence to wound. It is also shown that the mice have no restrictions during the post operative period. Dressings are removed 7-12 days later. The silicone protective layer is easily peeled off once the wound has re-epithelialized.

FIGS. 6A-C show macroscopic evidence of hair growth as soon as dressings are removed on day 11 (A) and full growth of hair over grafted region by day 21 (B-C).

FIG. 7 shows markers of hair development during early stages of hair reconstitution. H&E staining reveals that cells start at the base of the scaffold near the wound bed and migrate to the surface as the cells differentiate and organize themselves into pilosebaceous units within normal skin. β-catenin: First evidence begins on day 7 scattered throughout the cells within the matrix. Cells quickly organize themselves by the next day to form the beginnings of hair. K14: There is evidence of basal keratinocytes scattered throughout the matrix initially. They then organize themselves into a basal epidermal layer. NCAM: Positive cells organize themselves over the course of time to the subepidermal layer. Involucrin: Positive cells organize themselves into the basal epidermal layer reconstituting normal epidermis and hair shaft. Versican: Positive cells begin in the same layer as all other cells and by day 8 have homed to the dermal papilla. Note that hair follicle orientation is then readjusted toward the epidermal interface.

FIGS. 8A-E show wound healing and regeneration. (A) Hairs continue to grow over one year after grafting shown most evidently after shaving. (B-E) Post wound regeneration of hairs is shown after hair grows back after being plucked.

FIGS. 9A-C illustrate different ways of patterning skin stem cells. A population of skin stem cell (A) can generate numerous small hairs (B) or fewer larger hairs (C), depending on environmental conditions.

FIG. 10 illustrates the composition of a viral vector which can be used in combination with packaging and pseudotyping construct to generate lentivirus useful for molecular reprogramming.

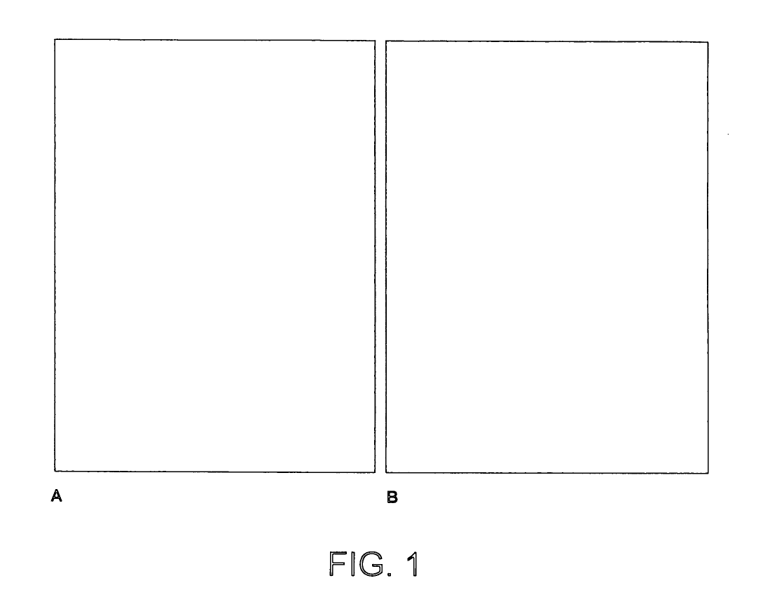

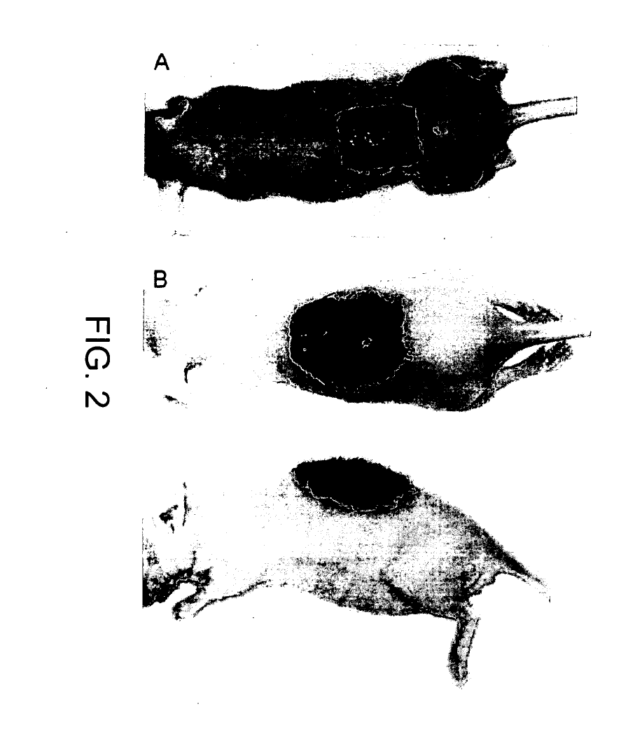

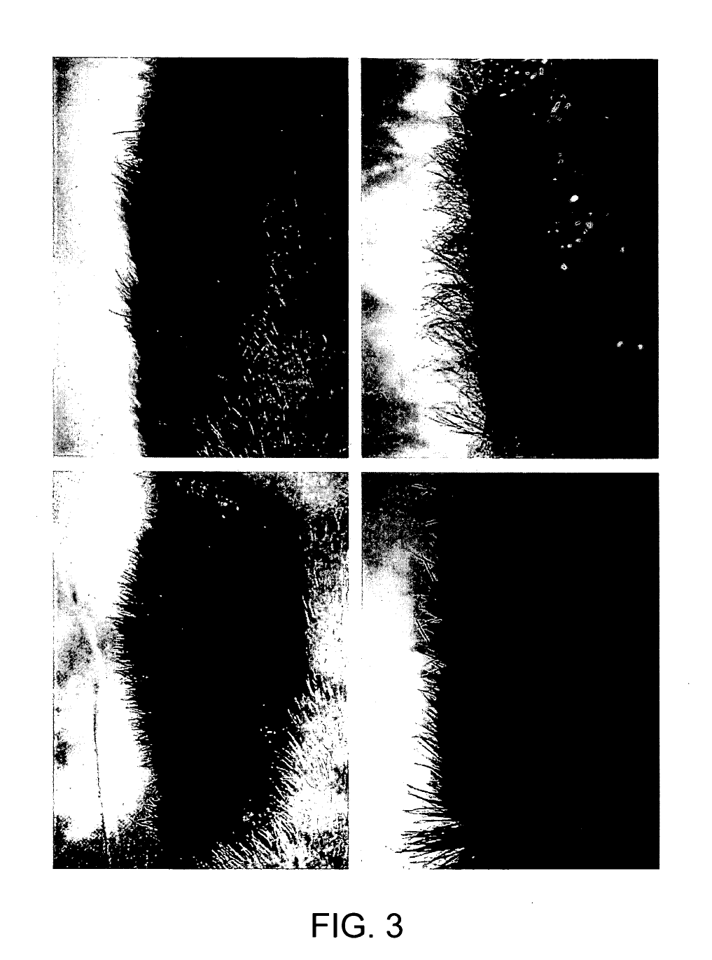

### DETAILED DESCRIPTION OF THE INVENTION

Throughout this disclosure, various publications, patents and published patent specifications are referenced by an identifying citation. Also within this disclosure are Arabic numerals referring to referenced citations, the full bibliographic details of which are provided immediately preceding the claims. The disclosures of these publications, patents and published patent specifications are hereby incorporated by reference in their entirety into the present disclosure to more fully describe the state of the art to which this invention pertains.

### DEFINITIONS

All numerical designations, e.g., pH, temperature, time, concentration, and molecular weight, including ranges, are approximations which are varied (+) or (−) by increments of 1.0 or 0.1, as appropriate. It is to be understood, although not always explicitly stated that all numerical designations are preceded by the term “about”. It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a cell” includes a plurality of cells, including mixtures thereof.

As used herein, the term “comprising” is intended to mean that the compositions and methods include the recited elements, but not excluding others. “Consisting essentially of when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of shall mean excluding more than trace elements of other ingredients and substantial method steps for administering the compositions of this invention or process steps to produce a composition or achieve an intended result. Embodiments defined by each of these transition terms are within the scope of this invention.

The term “isolated” or “purified” means separated from constituents, cellular and otherwise, in which the cell, tissue, polynucleotide, peptide, polypeptide, protein, antibody or fragment(s) thereof, which are normally associated in nature. For example, an isolated polynucleotide is separated from the 3′ and 5′ contiguous nucleotides with which it is normally associated in its native or natural environment, e.g., on the chromosome. As is apparent to those of skill in the art, a non-naturally occurring polynucleotide, peptide, polypeptide, protein, antibody or fragment(s) thereof, does not require “isolation” to distinguish it from its naturally occurring counterpart. An isolated cell is a cell that is separated form tissue or cells of dissimilar phenotype or genotype.

As used herein, the term “Pilosebaceous Unit” refers to the structure present on the surface of mammalian skin consisting of hair follicle, hair shaft and sebaceous gland. Pilosebaceous units are considered as an important pathway for percutaneous absorption of topically applied drugs and delivery systems. Pilosebaceous units are also the structural units for hair growth. For structural and functional descriptions of pilosebaceous units, see Singh et al. (2000) Indian J. Pharmacol. 32:269-281.

As used herein, the term “physiological plane” or “topological plane” refers to the physiological orientation of hair growth, in which the hairs grow towards the outside of the skin of the subject rather than on the underside resulting in formation of cysts.

As used herein, “stem cell” defines a cell with the ability to divide for indefinite periods in culture and give rise to specialized cells. At this time and for convenience, stem cells are categorized as somatic (adult) or embryonic. A somatic stem cell is an undifferentiated cell found in a differentiated tissue that can renew itself (clonal) and (with certain limitations) differentiate to yield all the specialized cell types of the tissue from which it originated. An embryonic stem cell is a primitive (undifferentiated) cell from the embryo that has the potential to become a wide variety of specialized cell types. An embryonic stem cell is one that has been cultured under in vitro conditions that allow proliferation without differentiation for months to years. Non-limiting examples of embryonic stem cells are the HES2 (also known as ES02) cell line available from ESI, Singapore and the H1 (also know as WA01) cell line available from WiCells, Madison, Wis. Pluripotent embryonic stem cells can be distinguished from other types of cells by the use of markers including, but not limited to, Oct-4, alkaline phosphatase, CD30, TDGF-1, GCTM-2, Genesis, Germ cell nuclear factor, SSEA1, SSEA3, and SSEA4.

A clone is a line of cells that is genetically identical to the originating cell; in this case, a stem cell. “Clonal proliferation” refers to the growth of a population of cells by the continuous division of single cells into two identical daughter cells and/or population of identical cells.

A “precursor” or “progenitor cell” intends to mean cells that have a capacity to differentiate into a specific type of cell. A progenitor cell may be a stem cell. A progenitor cell may also be more specific than a stem cell. A progenitor cell may be unipotent or multipotent. Compared to adult stem cells, a progenitor cell may be in a farther stage of cell differentiation. Progenitor cells are often found in adult organisms, they act as a repair system for the body. Examples of progenitor cells include, but are not limited to, satellite cells found in muscles, intermediate progenitor cells formed in the subventricular zone, bone marrow stromal cells, periosteum progenitor cells, pancreatic progenitor cells and angioblasts or endothelial progenitor cells. Examples of progenitor cells may also include, but are not limited to, epidermal and dermal cells from neonatal organisms.

As used herein, a “pluripotent cell” defines a less differentiated cell that can give rise to at least two distinct (genotypically and/or phenotypically) further differentiated progeny cells. In another aspect, a “pluripotent cell” includes a Induced Pluripotent Stem Cell (iPSC) which is an artificially derived stem cell from a non-pluripotent cell, typically an adult somatic cell, produced by inducing expression of one or more stem cell specific genes Such stem cell specific genes include, but are not limited to, the family of octamer transcription factors, i.e. Oct-3/4; the family of Sox genes, i.e. Sox1, Sox2, Sox3, Sox 15 and Sox 18; the family of Klf genes, i.e. Klf1, Klf2, Klf4 and Klf5; the family of Myc genes, i.e. c-myc and L-myc; the family of Nanog genes, i.e. OCT4, NANOG and REX1; or LIN28. Examples of iPSCs are described in Takahashi et al. Cell advance online publication 20 Nov. 2007 131(5):861-72, 2007; Takahashi & Yamanaka Cell 126:663-76, 2006; Okita et al. Nature 448:260-262, 2007; Yu et al. Science advance online publication 20 Nov. 2007 318(5858):1917-20, 2007; and Nakagawa et al. Nat Biotechnol. Advance online publication 30 Nov. 2007 26(1):101-6, 2008.

A “multi-lineage stem cell” or “multipotent stem cell” refers to a stem cell that reproduces itself and at least two further differentiated progeny cells from distinct developmental lineages. The lineages can be from the same germ layer (i.e. mesoderm, ectoderm or endoderm), or from different germ layers. An example of two progeny cells with distinct developmental lineages from differentiation of a multilineage stem cell is a myogenic cell and an adipogenic cell (both are of mesodermal origin, yet give rise to different tissues). Another example is a neurogenic cell (of ectodermal origin) and adipogenic cell (of mesodermal origin).

A skin precursor cell intends a pluripotent stem or progenitor cell with the ability to differentiate into at least one of epidermal, dermal and hair tissue types. A multipotent skin precursor cell is identified by one or more markers such as sca-1, fibronectin, p63, S100A6, keratin 19 (K19), SOX2 or β₁ integrin.

An “epidermal precursor cell” as used herein intends cells having the potential to differentiate into epidermal cells. Typically, these cells are identified by the marker β₁ integrin.

A “dermal precursor cell” as used herein intends cells having the potential to differentiate into dermal cells. Typically these cells are identified by one or more of the markers p63, S100A6 or β₁ integrin.

The term “propagate” means to grow or alter the phenotype of a cell or population of cells. The term “growing” refers to the proliferation of cells in the presence of supporting media, nutrients, growth factors, support cells, or any chemical or biological compound necessary for obtaining the desired number of cells or cell type. In one embodiment, the growing of cells results in the regeneration of tissue.

The term “culturing” refers to the in vitro propagation of cells or organisms on or in media of various kinds. It is understood that the descendants of a cell grown in culture may not be completely identical (i.e., morphologically, genetically, or phenotypically) to the parent cell. By “expanded” is meant any proliferation or division of cells.

As used herein, the “lineage” of a cell defines the heredity of the cell, i.e. its predecessors and progeny. The lineage of a cell places the cell within a hereditary scheme of development and differentiation.

A derivative of a cell or population of cells is a daughter cell of the isolated cell or population of cells. Derivatives include the expanded clonal cells or differentiated cells cultured and propagated from the isolated stem cell or population of stem cells. Derivatives also include already derived stem cells or population of stem cells.

“Differentiation” describes the process whereby an unspecialized cell acquires the features of a specialized cell such as a heart, liver, or muscle cell. “Directed differentiation” refers to the manipulation of stem cell culture conditions to induce differentiation into a particular cell type. “Dedifferentiated” defines a cell that reverts to a less committed position within the lineage of a cell. As used herein, the term “differentiates or differentiated” defines a cell that takes on a more committed (“differentiated”) position within the lineage of a cell. As used herein, “a cell that differentiates into a mesodermal (or ectodermal or endodermal) lineage” defines a cell that becomes committed to a specific mesodermal, ectodermal or endodermal lineage, respectively. Examples of cells that differentiate into a mesodermal lineage or give rise to specific mesodermal cells include, but are not limited to, cells that are adipogenic, leiomyogenic, chondrogenic, cardiogenic, dermatogenic, hematopoetic, hemangiogenic, myogenic, nephrogenic, urogenitogenic, osteogenic, pericardiogenic, or stromal.

Examples of cells that differentiate into ectodermal lineage include, but are not limited to epidermal cells, neurogenic cells, and neurogliagenic cells.

Examples of cells that differentiate into endodermal lineage include, but are not limited to pleurogenic cells, and hepatogenic cells, cell that give rise to the lining of the intestine, and cells that give rise to pancreogenic and splanchogenic cells.

The term “neonatal” intends a newborn mammal. In one aspect, a neonatal human is a human infant during the first month after birth. An “aged” mammal refers to an grown up or adult mammal.

“Bone Morphogenic Proteins” (BMP) are a group of multifunctional growth factors and cytokines with effects in various tissues. For example, BMPs are known to induce the formation of bone and/or cartilage. Examples of BMP may include, but are not limited to BMP1, BMP2, BMP3, BMP4, BMP5, BMP6, BMP7, BMP8a, BMP8b, BMP10 and BMP15.

“BMP signaling” or “BMP signaling pathway” refers to the enzyme linked receptor protein signaling transduction pathway involving proteins that directly or indirectly regulate (activate or inhibit) downstream protein activity or gene expression. Examples of molecules involved in the BMP signaling pathways may be found in the public Gene Ontology (GO) database, under GO ID: GO:0030509, accessible at the web page (amigo.geneontology.org/cgi-bin/amigo/term-details.cgi?term=GO:0030509&session_id=5573amigo1226631957), last accessed on Nov. 17, 2008. Without limitation, examples of proteins in the BMP signaling pathway include Activin receptor type-1 (ACVR1, UniProt: Q04771), Activin receptor type-2A (ACVR2A, UniProt: P27037), Activin receptor type-2B (ACVR2B, UniProt: Q13705), BMP1 (UniProt: P13497), BMP2 (UniProt: P12643), BMP3 (UniProt: P12645), BMP4 (UniProt: P12644), BMP5 (UniProt: P22003), BMP6 (UniProt: P22004), BMP7 (UniProt: P18075), BMP8a (UniProt: Q7Z5Y6), BMP8b (UniProt: P34820), BMP10 (UniProt: 095393), BMP15 (UniProt: 095972), Bone morphogenetic protein receptor type-1A (BMPR1A, UniProt: P36894), Bone morphogenetic protein receptor type-1B (BMPR1B, UniProt: 000238), Bone morphogenetic protein receptor type-2 (BMPR2, UniProt: Q13873), Chordin-like protein (CHRDL1, UniProt: Q9BU40), Follistatin-related protein 1 (FSTL1, UniProt: Q12841), Growth/differentiation factor 2 (GDF2, UniProt: Q9UK05), Growth/differentiation factor 6 (GDF6, UniProt: Q6KF10), Growth/differentiation factor 7 (GDF7, UniProt: Q7Z4P5), Gremlin-2 (GREM2, UniProt: Q9H772), RGM domain family member B (RGMB, UniProt: Q6NW40), Ski oncogene (SKI, UniProt: P12755), Mothers against decapentaplegic homolog 4 (SMAD4, UniProt: Q13485), Mothers against decapentaplegic homolog 5 (SMAD5, UniProt: Q99717), Mothers against decapentaplegic homolog 6 (SMAD6, UniProt: O43541), Mothers against decapentaplegic homolog 7 (SMAD7, UniProt: O15105), Mothers against decapentaplegic homolog 9 (SMAD9, UniProt: O15198), E3 ubiquitin-protein ligase SMRF2 (SMURF2, UniProt: Q9HAU4), TGF-beta receptor type III (TGFBR3, UniProt: Q03167), Ubiquitin-conjugating enzyme E2 D1 (UBE2D1, UniProt: P51668), Ubiquitin-conjugating enzyme E2 D3 (UBE2D3, UniProt: P61077) and Zinc finger FYVE domain-containing protein 16 (ZFYVE16, UniProt: Q7Z3T8). Proteins that positively or negatively regulate the BMP signaling, for purpose of this invention, are also considered within the meaning of the BMP signaling. Proteins that positively regulate BMP signaling include, but are not limited to, Serine/threonine-protein kinase receptor R3 (ACVRL1, UniProt: P37023) and Endoglin (ENG, UniProt: P17813). Proteins that negatively regulate BMP signaling include, but are not limited to, Chordin (CHRD, UniProt: Q9H2X0), E3 ubiquitin-protein ligase SMURF1 (SMURF1, UniProt: Q9HCE7), Sclerostin (SOST, UniProt: Q9BQB4) and Brorin (VWC2, UniProt: Q2TAL6). Examples of proteins in the BMP signaling pathway may also include Proprotein convertase subtilisin/kexin type 6 (PCSK6, UniProt: P29122) that regulates BMP signaling.

Small molecules, polynucleotides, polypeptides that enhance or inhibit BMP signaling exist or can be made with procedures known by those skilled in the art. Yanagita (2009) BioFactors 35(2):113-199 is a review article discussing BMP regulators (incorporated by reference). For example, dorsomorphin is a potent small molecule BMP antagonist (Hao et al. (2008) PLoS ONE, 3(8):e2904, Yu et al. (2008) Nat Chem Biol. 4(1):33-41). Dorsomorphin is currently commercially available from several vendors. Dorsomorphin was reported to selectively inhibit the BMP receptors, type I: ALK2, ALK3 and ALK6 and thus “blocks BMP-mediated SMAD1/5/8 phosphorylation”. Dorsomorphin has preferential specificity toward inhibiting BMP versus TGF-beta and activin signaling. In published reports, dorsomorphin is characterized by low toxicity and it can be delivered into skin to lower macro-environmental BMP signaling and create favorable conditions for hair growth to occur. A unique property of dorsomorphin is that it is a small molecule and is soluble in DMSO. DMSO is known to significantly facilitate trans-dermal delivery of small molecule drugs. This enhancing effect of DMSO on skin penetration can be used in non-invasive method of pharmacological modulation of dermal macro-environment. Treatment procedure thus consists of simply applying liquid form of dorsomorphin in DMSO onto the surface of intact skin. Dorsomorphin in DMSO can be made in form of cream that can be simply rubbed onto intact skin. Small molecule agonist and antagonists for other signaling pathways also exist and can be used to augment or inhibit BMP signaling. Interaction of these small molecules with pathways including, but not limited to, WNT, SHH and FGF will also have direct or indirect impact on BMP signaling thus serve as effective modulator of hair growth via methods disclosed in this invention.

Other types of BMP agonists or antagonists also exist. Yanagita (2009) BioFactors 35(2):113-199 is a review article discussing BMP regulators (incorporated by reference). Non-limiting examples include such as noggin, chordin, gremlin, sclerostin and follistatin. Representative sequences for these proteins include UniProt: Q13253 for noggin, UniProt: Q9H2X0 for chordin, UniProt: 060565 for gremlin, UniProt: Q9BQB4 for sclerostin, and UniProt: P19883 for follistatin. Noggin (UniProt: Q13253), for example, can be produced using methods described in, e.g. McMahon et al. (1998) Genes & Development 12:1438-52.

In some aspects, an agent that can augment or inhibit BMP signaling is a small molecule agonist or antagonist to a BMP agonist or antagonist. In one aspect, the small molecule is a noggin agonist. In another aspect, the small molecule is a noggin antagonist.

Examples of agents that can augment or inhibit BMP signaling also include, but are not limited to, polynucleotides that encode BMP proteins, encode polypeptides augmenting or inhibiting BMP signaling, or augmenting or inhibit expression of BMP proteins, or polypeptides augmenting or inhibiting BMP signaling. In some embodiments, the agent is small interference RNA (siRNA) or double strand RNA (dsRNA) that inhibits expression of proteins that augment or inhibit BMP signaling.

Examples of agents that can augment or inhibit BMP signaling may also include, but are not limited to, an isolated or recombinant BMP protein, or isolated or recombinant polypeptide enhancing or inhibiting BMP signaling. In some aspect, the agent further comprises a pharmaceutically acceptable carrier. In another aspect, the compositions contain carriers that modulate (controlled release) the release of the active agent when administered to a subject in need thereof.

Examples of polypeptide agents that augment or inhibit BMP signaling may also include, but are not limited to, antibodies or modified antibodies including, but not limited to, blocking fragments of antibodies, that activate, stabilize or inhibit proteins in the BMP signaling pathway or proteins modulating the BMP signaling pathway, thereby augmenting or inhibiting BMP signaling.

As used herein, the term “modulate” refers to an act by an agent to regulate, to control or to change certain characteristics of the formation of pilosebaceous units. Examples of the agent may include, but are not limited to, proteins or polypeptides, DNA, RNA, siRNA, dsRNA or other polynucleotides, small molecules. The agent may also mean a temperature change, physical movement or stimulus or any other therapeutic or clinical means that alter the formation of pilosebaceous units. Without limitation, the object may mean a biochemical molecule or pathway, a biochemical activity, a medical condition or any other chemical, biochemical, physical or medical aspect of a subject. In one aspect, the term “modulate” means to enhance the formation of pilosebaceuous units in a plane. In another aspect, the term “modulate” means to inhibit the formation of pilosebaceous units on a plane.

The terms “inhibit” or “antagonize” intend mean an decrease of amount or formation of pilosebaceous units on a plane.

An “agonist”, as used herein, refers to a drug or other chemical that can bind a receptor on a cell to produce a physiologic reaction typical of a naturally occurring substance. The efficacy of an agonist may be positive, causing an increase in the receptor's activity or negative causing a decrease in the receptor's activity.

An “antagonist” refers to a type of receptor ligand or drug that does not provoke a biological response itself upon binding to the receptor, but blocks or dampens agonist-mediated responses. In pharmacology, antagonists have affinity but no efficacy for their cognate receptors and binding will disrupt the interaction and inhibit the function of an agonist or inverse agonist at receptors. Antagonists mediate their effects by binding to the active site or to allosteric sites on receptors or they may interact at unique binding sites not normally involved in the biological regulation of the receptor's activity. Antagonist activity may be reversible or irreversible depending on the longevity of the antagonist-receptor complex which in turn depends on the nature of antagonist receptor binding. The majority of drug antagonists achieve their potency by competing with endogenous ligands or substrates at structurally defined binding sites on receptors.

The term “hair growth” intends to include, but not limited to, the formation of new hair or growth of existing hair.

“Spironolactone” (IUPAC name: 7α-Acetylthio-3-oxo-17α-pregn-4-ene-21,17-carbolactone is marketed under the trade names Aldactone, Novo-Spiroton, Aldactazide, Spiractin, Spirotone, Verospiron or Berlactone) is a diuretic and is used as an antiandrogen. It is also used for treating hair loss in women, and can be used as a topical medication for treatment of male baldness.

“Minoxidil” (trade names Rogaine and Regaine; IUPAC name: 6-piperidin-1-ylpyrimidine-2,4-diamine 3-oxide) is a commercially available topical formulation that inhibits hair loss is a vasodilator medication that is available over the counter for treatment of androgenic alopecia, among other baldness treatments.

“Finasteride” (IUPAC name N-(1,1-dimethylethyl)-3-oxo-(5α,17β)-4-azaandrost-1-ene-17-carboxamide) is a synthetic antiandrogen that acts by inhibiting type II 5-alpha reductase, the enzyme that converts testosterone to dihydrotestosterone (DHT). It is used to treat prostate cancer and is registered in many countries to treat adrogenetic alopecia or male pattern baldness. “Propecia” is a medicament containing finasteride as an active ingredient is commercially available from Merck & Co., Inc.

“Administration”, as used herein, refers to the delivery of a medication or matrix composition to a mammal or subject to be treated and/or in need of such treatment. Non-limiting examples include oral dosing, intracutaneous injection, direct application to target area proximal areas on the skin, or applied on a patch. Various physical and/or mechanical technologies are available to permit the sustained or immediate topical or transdermal administration of macromolecules (such as, peptides). Such technologies include iontophoresis (see for example Kalia et al. (2004) Adv. Drug Del. Rev. 56:619-58) sonophoresis, needle-less injection, and/or microstructured arrays (sometimes called microneedles; one particular example is the Microstructured Transdermal System (MTS) commercially available from 3M) (see, e.g., Alain et al. (2002) J. Control. Release 81:113-119; Santi et al. (1997) Pharm. Res. 14(1):63-66; Sebastien et al. (1998) J. Pharm. Sci. 87(8):922-925). Methods of making and using arrays of solid microneedles that can be inserted into the skin for transdermal delivery of peptides (such as cyclic CRF antagonists) are provided in Martanto et al. (2004) Pharm. Res. 21:947-52, and Martano et al. (2005) Am. Inst. Chem. Eng. 51:1599-607. In some examples, the delivery system includes a combination of systems, such as microneedles made of biocompatible and biodegradable polymers (Park et al. (2005) J. Control. Release 104:51-66). In one aspect, administration is topical administration as defined herein.

“Topical administration” refers to delivery of a composition or medication by application to the skin. Non-limiting examples of topical administration include any methods described under the definition of “administration” pertaining to delivery of a medication to the skin.

A “composition” is intended to mean a combination of active agent, cell or population of cells and another compound or composition, inert (for example, a detectable agent or label or biocompatible scaffold) or active, such as a growth and/or differentiation factor.

A “pharmaceutical composition” is intended to include the combination of an active agent with a carrier, inert or active such as a biocompatible scaffold, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, Remington's Pharm. Sci., 15th Ed. (Mack Publ. Co., Easton (1975)). The term includes carriers that facilitate controlled release of the active agent as well as immediate release.

For topical use, the pharmaceutically acceptable carrier is suitable for manufacture of creams, ointments, jellies, gels, solutions, suspensions, etc. Such carriers are conventional in the art, e.g., for topical administration with polyethylene glycol (PEG). These formulations may optionally comprise additional pharmaceutically acceptable ingredients such as diluents, stabilizers, and/or adjuvants.

The pharmaceutically acceptable carrier facilitate immediate or controlled release of the active ingredient.

“An effective amount” refers to the amount of cells or a biological or chemical agent sufficient to induce a desired biological and/or therapeutic result. That result can be alleviation of the signs, symptoms, or causes of a disease, or any other desired alteration of a biological system. In one aspect, the result of an effective amount of skin precursor cells can include generation of pilosebaceous unites in a physiologically plane. In another aspect, the result of an effective amount of an agent inhibiting the BMP signaling can be inhibition of BMP signaling. In yet another aspect, an effective amount of an a gene promoting cell differentiation can be promotion of cell differentiation. The effective amount will vary depending upon the specific cell type or agents used, the desired size or stage of the generated pilosebaceous units, the manner of administration and the like, all of which can be determined readily by one of ordinary skill in the art.

An “epithelial sheet” refers to a biological dressing composed of epidermal keratinocytes and formed in culture as three-dimensional sheet have which has been used for wound healing as skin grafts (See, e.g., U.S. Pat. Nos. 5,292,655, 5,686,307, 5,834,312, 5,912,175, 6,162,643, and 7,037,721).

A “subject” of diagnosis or treatment is a cell or a mammal, including a human. Non-human animals subject to diagnosis or treatment include, for example, murine, such as rats, mice, canine, such as dogs, leporids, such as rabbits, bovine, simian, ovine, livestock, sport animals, and pets.

### MODES FOR CARRYING OUT THE INVENTION

Thus, in one aspect this invention provides a composition useful in one aspect to generate pilosebaceous units in a physiological plane comprising, or alternatively consisting essentially of, or yet further consisting of, a biocompatible scaffold and an effective amount of skin precursor cells contained within or on the matrix.

In one aspect, the skin precursor cells comprise, or alternatively consist essentially of, or yet further consist of, stem cells, epidermal precursor cells or dermal precursor cells. Stem cells, epidermal and/or dermal precursor cells can be of any appropriate type, e.g., an animal such as a mammal, including a human. Non-human animals include, for example, murine (such as rats or mice), canine, such as dogs, leporids, such as rabbits, equine, bovine, simian, livestock, sport animals, and pets. In one aspect, the cell species type is selected for compatibility with the host into which the composition is implanted, e.g., murine for a murine host and human for a human host.

Epidermal precursor cells can be isolated from animal or human keritoncytes and selected as described herein or in Fortunel et al. (2003) J. Cell Science 118:4043-4052. In one embodiment, the epidermal cells comprise keratinocyte stem cells, follicular papillae, sheath cells, non-stem cell keratinocytes, or any combination thereof.

Dermal precursor cells also can be isolated from non-human animals as described herein or from human sources as described in Medina et al. (2006) J. of Cellular Biochem. 98(1):174-84.

In a further aspect, adult or somatic stem cells can be utilized in the compositions of this invention. Typically, the cells are identified by the stem cells markers and can be isolated using the methods as described by, e.g., Reiisi (2009) In Vitro Cell Dev. Biol. Anim. 2009 Nov. 14 [Epub ahead of print].

In one aspect the cells are allogeneic to the subject. In another aspect, the cells are autologous. In a further aspect, the cells are a mixture of allogeneic and autologous.

In a further aspect, the compositions comprises, or alternatively consists essentially of or yet further consists of a combination of dermal precursor cells, epidermal precursor cells and stem cells, e.g. one or more of adult or somatic stem cells, embryonic stem cells and iPS cells.

The cells are combined with a biocompatible matrix. As used herein, the term intends a compositions that has the ability to support cell growth either in vitro or in vivo, the ability to support the growth of pilosebaceous units, the ability to be endowed with varying degrees of flexibility or rigidity required, the ability to have varying degrees of biodegradability, the ability to be introduced into the intended site in vivo without provoking secondary damage, and the ability to serve as a vehicle or reservoir for delivery of drugs or bioactive substances to the desired site of action. Prior art matrices are known, and include for example gels, foams, sheets, and numerous porous particulate structures of different forms and shapes.

The matrix can be composed of biopolymers, including polypeptides or proteins, as well as various polysaccharides, including proteoglycans and the like. In addition, these biopolymers may be either selected or manipulated in ways that affect their physico-chemical properties. For example biopolymers may be cross-linked either enzymatically, chemically or by other means, thereby providing greater or lesser degrees of rigidity or susceptibility to degradation. Natural polymers which have been disclosed to be useful for tissue engineering or culture, one can enumerate various constituents of the extracellular matrix including hydrogels, fibronectin, various types of collagen, and laminin, as well as keratin, fibrin and fibrinogen, hyaluronic acid, heparin sulfate, chondroitin sulfate and others. These are described for example in U.S. Patent Publ. No. 2005/0260753, U.S. Pat. Nos. 7,452,720; 4,829,000; 5,942,499; 5,128,326; 5,783,691; 5,955,438; 4,971,954; 5,948,429; 6,083,383; 5,411,885; 5,279,825; 5,173,295; 4,642,120; 6,124,265 and 6,110,487.

A biocompatible matrix includes acellular matrices that have hair enhancing activity which can be prepared according to methods described in the literature, e.g., Schedin et al. (2004) Oncogene. 23(9):1766-79 and Potapova et al. (2008) Am. J. Physiol. Heart Circ. Physiol. 295(6):H2257-63, from E13 mouse dermis which has hair enhancing ability.

Commercially available matrices are also useful, examples of which include, without limitation FDA approved Integra or Alloderm matrices and commercially available matrices described in U.S. Pat. No. 7,452,720. Such matrices include EpiCel™, Integra™, AlloDerm™, DermaGraft™, Hyaff/LaserSkin™, and PolyActive™. Materials to either temporarily cover wounds, or to stimulate permanent skin repair processes, included ApliGraft™, Comp Cult Skin™, OrCel™, TransCyte™ and BioBrane™.

In a further aspect, the composition further comprises a detectable marker or label to monitor growth and differentiation of the cells. Examples of such include for example, luciferase under the control of a ubiquitin promoter, GFP, herpes simplex virus type 1 thymidine kinase (HSV-1 TK) under the control of a ubiquitin promoter and super-paramegnetic iron oxide (SPIO) nanoparticles. These systems are useful to detect teratoma formation or anomalous skin structures.

In one aspect of this invention, the composition may be prepared by admixing an effective amount of isolated skin precursor cells in serum-free medium and a biocompatible scaffold, under conditions that favor the incorporation of the cells into the biocompatible scaffold. In some embodiments, the concentration of the cells in the scaffold is from about 800,000 cells/mm³ to 1,500,000 cells/mm³. In some embodiments, the scaffold and cells are admixed by passively contacting the cells with the scaffold at a temperature range from about 25 to about 37° C. for about 30 minutes to 2 hours.

The compositions can alternatively contain an effective amount of differentiation or growth factor that promotes cell differentiation or growth. Non-limiting examples of such factors include agents that inhibit Born Morphogenic Protein (BMP) signaling, such as noggin (UniProt: Q13253) which can be produced using methods described in, e.g. McMahon et al. (1998) Genes & Development, 12:1438-52, chordin, gremlin, sclerostin and follistatin and any combination thereof. Use of the terms such as “growth factors, cytokines, hormones” is to be exemplary. In one embodiment, the factor comprises Platelet Derived Growth Factor (PDGF) available from R&D Ssytems, Minneapolis, Minn., Vascular Endothelial Growth Factor (VEGF) available from Abcam, Cambridge, Mass., Epithelial Growth Factor (EGF) available from Abcam, Cambridge, Mass., TGF-β available from Abcam, Cambridge, Mass., Fibroblast Growth Factor (FGF), insulin available from Abcam, Cambridge, Mass., transferrin, retinoid, or any combination thereof. In another embodiment, the composition is suitable for culturing mammalian epidermal cells and therefore can comprise cell culture medium as known to those of skill in the art, e.g., without limitation serum-free medium commercially available from Invitrogen (Carlsbad, Calif.). Additional components are optionally added to the composition, that include, but are not limited to antibiotics, albumin, amino acids, and other components known to the art for the culture of cells. Additionally, components optionally are added to enhance the differentiation process. Effective amounts of the differentiation and/or growth factors can be empirically determined by those of skill in the art. It is appreciated that such amounts will vary with the source of the cells, the ultimate composition (differentiated cell type(s)) desired after culturing or the differentiation of the cells and/or growth factors and the ultimate utility for the composition. An effective amount for an in vitro screen will not necessarily be the same as when the composition is to be administered to an animal such as a human patient.

The compositions can alternatively contain an effective amount of minoxidil (commercially available under the trademark “Rogaine” (Pharmacia & Upjohn Company)), finasteride or other agent that enhances hair growth.

The invention also provides compositions comprising the serum-free medium described above, wherein the medium comprises reduced concentrations of one or more factors that modulate cell growth. In one embodiment, the factor comprises PDGF, VEGF, EGF, TGF-β, FGF, insulin, transferrin, retinoid, or any combination thereof. In another embodiment, the medium is suitable for culturing mammalian (e.g., murine, rat or human) epidermal cells. In another embodiment, the culturing comprises cell differentiation. In one embodiment, the epidermal cells comprise keratinocyte stem cells, follicular papillae, sheath cells, non-stem cell keratinocytes, or any combination thereof.

In the above embodiments, the concentration of cells in the scaffold is from about 800,000 cells/mm³ to about 1,500,000 cells/mm³. In some aspects, the concentration of cells in the scaffold is equal to or greater than about 10,000 cells/mm³, or alternatively is equal to or greater than about 50,000 cells/mm³, or alternatively is equal to or greater than about 100,000 cells/mm³, or alternatively is equal to or greater than about 200,000 cells/mm³, or alternatively is equal to or greater than about 300,000 cells/mm³, or alternatively is equal to or greater than about 400,000 cells/mm³, or alternatively is equal to or greater than about 500,000 cells/mm³, or alternatively is equal to or greater than about 600,000 cells/mm³, or alternatively is equal to or greater than about 700,000 cells/mm³, or alternatively is equal to or greater than about 800,000 cells/mm³, or alternatively is equal to or greater than about 900,000 cells/mm³, or or alternatively is equal to or greater than about 1,000,000 cells/mm³. In some aspects, the concentration of cells in the scaffold is equal to or less than about 800,000 cells/mm³, or alternatively is equal to or less than about 900,000 cells/mm³, or alternatively is equal to or less than about 1,000,000 cells/mm³, or alternatively is equal to or less than about 1,100,000 cells/mm³, or alternatively is equal to or less than about 1,200,000 cells/mm³, or alternatively is equal to or less than about 1,300,000 cells/mm³, or alternatively is equal to or less than about 1,400,000 cells/mm³, or alternatively is equal to or less than about 1,500,000 cells/mm³, or alternatively is equal to or less than about 1,600,000 cells/mm³, or alternatively is equal to or less than about 1,700,000 cells/mm³, or alternatively is equal to or less than about 1,800,000 cells/mm³, or alternatively is equal to or less than about 1,900,000 cells/mm³, or alternatively is equal to or less than about 2,000,000 cells/mm³, or alternatively is equal to or less than about 5,000,000 or or alternatively is equal to or less than about 10,000,000 cells/mm³. In one aspect, the skin precursor cells comprise epidermal and dermal precursor cells. In some embodiments, the ratio of epidermal to dermal precursor cells is about 2:1, or alternatively about 1:1, or alternatively about 1:2, or alternatively about 1:3, or alternatively about 1:4, or alternatively about 1:5, or alternatively about 1:6, or alternatively about 1:7, or alternatively about 1:8, or alternatively about 1:9, or alternatively about 1:10, or alternatively about 1:12, or alternatively about 1:15, or alternatively about 1:20 or alternatively about 1:50. In some aspects, the composition can further comprise, or alternatively consist essentially of, or yet further consist of, an effective amount of a suitable carrier and/or a growth or differentiation factor. In one aspect, the factor is selected from the group consisting of noggin, chordin, gremlin, sclerostin and follistatin and combinations thereof. In another aspect, the factor is selected from the group consisting of Platelet Derived Growth Factor (PDGF), Vascular Endothelial Growth Factor (VEGF), Epithelial Growth Factor (EGF), TGF-β, Fibroblast Growth Factor (FGF), insulin, transferrin, retinoid and combinations thereof.

In a further aspect, the composition further comprises a detectable marker or label to monitor growth and differentiation of the cells. Examples of such include for example, luciferase under the control of a ubiquitin promoter, GFP, herpes simplex virus type 1 thymidine kinase (HSV-1 TK) under the control of a ubiquitin promoter and super-paramegnetic iron oxide (SPIO) nanoparticles. These systems are useful to detect teratoma formation or anomalous skin structures.

In one aspect of this invention, the composition may be prepared by admixing an effective amount of isolated skin precursor cells in serum-free medium and a biocompatible scaffold, under conditions that favor the incorporation of the cells into the biocompatible scaffold. In some embodiments, serum-free media can support the maintenance and expansion of stem cells or precursor cells and various types of serum-free medic are commercially available from vendors. For examples, StemSpan® SFEM and StemSpan® H3000 are available from STEMCELL Technologies, Vancouver, BC, Canada. In some embodiments, the concentration of the cells to be admixed with the scaffold is an amount that will produce a concentration in the medium from about 800,000 cells/mm³ to about 1,500,000 cells/mm³. In some embodiments, the scaffold and cells are admixed by passively contacting the cells with the scaffold at a temperature range from about 25 to about 37° C. for about 30 minutes to about 2 hours. In one embodiment, the media containing the cells is merely placed on a surface of the scaffold.

In some embodiments, different ratios between the epidermal and dermal populations can be used to make the composition. As shown in Table 1, a ratio of epidermal and dermal precursor cells between about 1:5 and about 1:10 can be used to generate good pilosebaceous units. A combination of aged epidermal cells and newborn dermal cells, or a combination of newborn epidermal cells and aged dermal cells may not give rise to good hair growth. However, it has been noted that a replacement of newborn epidermal cells with aged epidermal cells had a lesser effect than a replacement of newborn dermal cells with aged dermal cells. Precursor cells can also be used to generate good hair growth. A combination of positive precursor cells and whole skin (WT) cells can lead to fair hair growth as well. While the use of Integra Matrix produced good hair growth, use of other scaffolds can result in good hair growth too.

This invention further provides a dermal patch comprising the compositions as noted above in combination with a dressing. A “dressing” refers to an overlay adjunct used by a mammal for application to a wound to promote healing and/or prevent further harm. A dressing may further comprise a bandage, which is primarily used to hold a dressing in place. In one aspect, a dressing can control the moisture content, protect the wound from infection, remove slough, or maintain the optimum pH or temperature to encourage healing. Non-limiting examples of dressings include a silicone protective layer or sheet, a collagen sheet, a plastic sheet or a latex sheet. In one aspect, the dressing is sterile. In a further aspect, the surface area of the dressing includes the entire area of the patch and extends beyond the periphery of the dermal patch and may optionally include an adhesive layer or coating around the periphery of the dressing but excluding the area of the patch. The adhesive coating or layer serves to secure the dermal patch to the situs of application. In a further aspect, the adhesive coating may exclude or include the area of the patch and if the adhesive coating includes the area of patch then the adhesive coating is irreversibly attached to the patch, or the adhesive coating can be reversible. In a yet further aspect, the dermal patch is stably attached to the dressing. In a yet further aspect, the dermal patch is removably attached to the dressing to allow for removing the patch overlay without removing the underlying patch.

Also provided by this invention is a method for generating pilosebaceous units in a physiological plane in a mammal in need thereof, comprising implanting the composition of the invention into the dermal layer of the subject such as a mammal under conditions that favor implantation of the composition into the dermis of the mammal. As used herein, mammals include, but are not limited to, murines, rats, simians, bovines, canines, humans, farm animals, sport animals and pets.

### Biocompatible Scaffolds

For the purpose of illustration only, examples of biocompatible scaffolds for use in this invention include, but are not limited to the porous and/or biodegradable and/or biocompatible scaffold as described in U.S. Pat. No. 4,947,840, col. 2, line 27 to col. 5, line 10, incorporated herein by reference in its entirety. In some other embodiments, a biocompatible scaffold is a dermal substitute consisting of amnion and biodegradable polymer as described in U.S. Patent Application Publication No. US 2005/0107876, paragraphs 28 to 64. In some other embodiments, a biocompatible scaffold is a single or double density biopolymer foam as described in International Patent Application Publication No. WO 98/22154, page 5, line 32 to page 23, line 33. In some other embodiments, a biocompatible scaffold is a gel-matrix-cells integrated system as described in International Patent Application Publication No. WO 2007/141028, page 13, line 1 to page 21, line 2. In some other embodiments, a biocompatible scaffold is a biomechanical implant as described in International Patent Application Publication No. WO 98/40111, page 7, line 13 to page 19, line 9.

In some embodiments, a biocompatible scaffold is a biocompatible nanofiber matrix as described in Venugopal et al. (2005) Tissue Engineering 11(5/6):847-54.

Examples of commercially available biocompatible scaffolds include, but are not limited to, Alloderm dermal collagen matrix (LifeCell Corporation, Branchburg, N.J.), Dermagraft-TC woven bioabsorbable polymer (polyglycolic and polylactic acids) membrane (Advanced Tissue Sciences, La Jolla, Calif.), Dermalogen human dermal collagen matrix (Collagenesis, Beverly, Mass.), Integra Bilayer Matrix Wound Dressing (Integra Life Sciences Corporation, Plainsboro, N.J.) and Fibrin Sealant Tisseel VH fibrin glue mixture (Baxter Health, Deerfield, Ill.). In some embodiments, the biocompatible scaffold can be type I collagen or silicon cell culture insert which are commercially available (e.g. Falcon™ Cell Culture Insert from BD Biosciences, San Jose, Calif.).

To make the composition, one admixes an effective amount of skin precursor cells in serum-free medium and a biocompatible scaffold, under conditions that favor the incorporation of the cells into the biocompatible scaffold. In one aspect, the resulted concentration of cells in the scaffold is from about 800,000 cells/mm³ to about 1,500,000 cells/mm³. In some aspects, the concentration of cells in the scaffold is equal to or greater than about 10,000 cells/mm³, or alternatively is equal to or greater than about 50,000 cells/mm³, or alternatively is equal to or greater than about 100,000 cells/mm³, or alternatively is equal to or greater than about 200,000 cells/mm³, or alternatively is equal to or greater than about 300,000 cells/mm³, or alternatively is equal to or greater than about 400,000 cells/mm³, or alternatively is equal to or greater than about 500,000 cells/mm³, or alternatively is equal to or greater than about 600,000 cells/mm³, or alternatively is equal to or greater than about 700,000 cells/mm³, or alternatively is equal to or greater than about 800,000 cells/mm³, or alternatively is equal to or greater than about 900,000 cells/mm³, or alternatively is equal to or greater than about 1,000,000 cells/mm³. In some aspects, the concentration of cells in the scaffold is equal to or less than about 800,000 cells/mm³, or alternatively is equal to or less than about 900,000 cells/mm³, or alternatively is equal to or less than about 1,000,000 cells/mm³, or alternatively is equal to or less than about 1,100,000 cells/mm³, or alternatively is equal to or less than about 1,200,000 cells/mm³, or alternatively is equal to or less than about 1,300,000 cells/mm³, or alternatively is equal to or less than about 1,400,000 cells/mm³, or alternatively is equal to or less than about 1,500,000 cells/mm³, or alternatively is equal to or less than about 1,600,000 cells/mm³, or alternatively is equal to or less than about 1,700,000 cells/mm³, or alternatively is equal to or less than about 1,800,000 cells/mm³, or alternatively is equal to or less than about 1,900,000 cells/mm³, or alternatively is equal to or less than about 2,000,000 cells/mm³, or alternatively is equal to or less than about 5,000,000 cells/mm³or alternatively is equal to or less than about 10,000,000 cells/mm³. In one aspect, the skin precursor cells comprise epidermal and dermal precursor cells. In some embodiments, the ratio of epidermal to dermal precursor cells is about 2:1, or alternatively about 1:1, or alternatively about 1:2, or alternatively about 1:3, or alternatively about 1:4, or alternatively about 1:5, or alternatively about 1:6, or alternatively about 1:7, or alternatively about 1:8, or alternatively about 1:9, or alternatively about 1:10, or alternatively about 1:12, or alternatively about 1:15, or alternatively about 1:20 or alternatively about 1:50. In another aspect, the admixing is performed by passively contacting the cells with the scaffold, such as by soaking the scaffold with the cell composition in a pharmaceutically acceptable carrier at a temperature of about 25 to about 37° C. In one aspect, the biocompatible scaffold is dried or lyophilized prior to admixing with the cells in serum-free medium. In a further aspect, the method comprises, or alternatively consists essentially of, or alternatively, consists of admixing an effective amount of a growth factor selected from the group consisting of Platelet Derived Growth Factor (PDGF), Vascular Endothelial Growth Factor (VEGF), Epithelial Growth Factor (EGF), TGF-β, Fibroblast Growth Factor (FGF), insulin, transferrin, retinoid and combinations thereof. The additional agents can be combined with the scaffold and/or with the cells at the same time (concurrently) or after combination of the scaffold and cells, or prior to admixing the scaffold and cells.

### Skin Precursor Cell Sources

In one aspect, the skin precursor cells comprise dermal and epidermal precursor cells. In another aspect, the precursor cells comprise progenitor cells from adult skin or other tissues containing stem cells. In another aspect, the precursor cells can be adult or embryonic stem cells having the ability to differentiate into hair follicles under appropriate culturing or growth conditions that are present in the micro- or macro-environment (see e.g. Yu et al. (2006) Am. J. Pathol. 168(6):1979-88).

In some aspects, the skin precursor cells are embryonic stem (ES) cells. ES cells have the potential to develop into different cell types. Attempts have been made to guide them toward a particular lineage with selected medium conditions, activating endogenous transcriptional factors (Pera & Trounson (2004) Development 131(22):5515-25), transfecting cells with specific transcriptional factors (Muller et al. (2000) FASEB J. 14(15):2540-8), or co-culturing them with cells capable of lineage induction (Kawasaki et al. (2000) Neuron 28(1):31-40). Several successful methods can guide mouse ES cells toward a keratinocyte lineage (Aberdam (2004) Int. J. Dev. Biol. 48(2-3):203-236; luchi et al. (2006) Proc. Natl. Acad. Sci. U. S. A. 103:1792-1797; Coraux et al. (2003) Curr. Biol. 13(10):849-853; Ji et al. (2006) Tissue Eng. 12(4):665-679).

In some aspects, the skin precursor cells are cells isolated from human tissues. In one aspect, the skin precursor cells are Foreskin cells isolated from young children. In another aspect, the precursor cells are from adult human tissues. In one of such aspects, the skin precursor cells are cells isolated from the patient in need of the treatment. One of the risks in using stem cells is immunologic rejection, which can be alleviated by using a patient's own cells. It is valuable to isolate or convert adult cells to multi-potential skin stem cells.

In some embodiments, the skin precursor cells can be isolated from adult mammalian skin, such as Skin-derived Precursors (SKP) cells (Toma et al. (2005) Stem Cells, 23(6):727-737), or those from adipose tissue or bone marrow. These adult cells can be converted or differentiated into hair forming cells with procedures described in e.g. Hunt et al. (2008) Stem Cells 26(1):163-72. In another aspect, small molecules such as those targeting genes in the BMP pathway and Wnt pathway may be used to convert adult skin cells into progenitor cells (see e.g. Plikus et al. (2008) Nature 451(17):340-345).

In some aspects, methods or compositions known in the art can be used to induce hair forming ability from cells. In one aspect, acellular matrix is used. Acellular matrix is prepared from mammalian tissues (Schedin et al. (2004) Oncogene. 23(9):1766-79; Potapova et al. (2008) Am. J. Physiol. Heart Circ. Physiol. 295(6):H2257-63). Candidate cells are seeded in an acellular matrix derived from E13 mouse skin which has strong hair inducing ability. Hair follicles can induced from proper candidate cells.

In another aspect, small molecules and growth factors are used to induce the hair forming capability in cells. These cells are pretreated with growth factors or small molecules. Selection of candidate growth factors or small molecules is based on literature or microarray gene profiling analysis. They can be tested with methods described herein.

In another aspect, the skin precursor cells are Induced Pluripotent Stem (iPS) cells generated from cells isolated from adult tissues such as the skin by altering the transcription profile in the adult cells (see Takahashi et al. (2007) Cell 131(5):861-872 and Yu et al. (2007) Science 318(5858):1917-1920). These iPS cells can be converted to hair forming dermal papilla when they are incubated with stem cells with hair forming epidermis. The iPS cells can be converted to hair forming epidermis when they are incubated with stem cells with hair forming dermis or cell free matrix. In another aspect, the skin precursor cells comprise human adult keratinocytes and fibroblast cells.

Sources of skin precursor cells can be tested experimentally. In one aspect, multi-potential epidermal or dermal stem cells can be tested experimentally. For example, newborn mouse skin cells can serve as the positive control. Human or mouse or other types of mammalian epidermal stem cell candidates are tested in combination with newborn mouse dermal cells. Human or mouse or other types of mammalian dermal stem cell candidates are tested in combination with newborn mouse epidermal cells. Candidate cells are evaluated with a three-tier assay system with a higher throughput type screening first, and then with two of the more rigorous tests for hair forming ability. Tier (i), Mixed aggregate assay. In this assay, tested cells are mixed and cultured in shaking gassed flasks. Cells interact and differentiation genes are induced when right interactions occur. While cells sort to a certain extent, they remain disorganized. This assay is good for high throughput screening of cell interactions. This assay is based on the early work of Moscona (1980) Prog Clin Biol Res. 42:171-88, has been successfully used by the inventors as described in Grumet et al. (1984) Proc Natl Acad Sci U S A 81(24):7989-7993, and a recent application of this principle to hair differentiation is reported in Havlickova et al. (2008) J Invest Dermatol. [Epub 2008 Aug. 26]. The ability of candidate cells to express hair follicle differentiation genes are tested with gene markers. The markers are screened by RT-PCR or immuno-staining or other technologies known in the art. Tier (ii) Patch assay (as described in Zheng et al. (2005) J. Invest. Dermatol. 124(5):867-876). In this assay, dermal and epidermal cells are mixed in a high density suspension and injected subcutaneously. This assay is used to test the ability of cells to form a hair follicle structure. The number of hair filaments formed can be quantified. However, hair cysts form on the underside of the skin thus the score has to be made on the underside of the skin exposed. Tier (iii) Planar hair forming assay, as disclosed in the specification. This assay evaluates the topology of the whole hair follicle population to see if they are properly or physiologically arranged.

In one aspect of this invention, the composition may be prepared by admixing an effective amount of isolated skin precursor cells in serum-free medium or other pharmaceutically acceptable carrier and a biocompatible scaffold, under conditions that favor the incorporation of the cells into the biocompatible scaffold. In some embodiments, the scaffold and cells are admixed by passively contacting the cells with the scaffold at a temperature range from about 25 to about 37° C. for about 30 minutes to about 2 hours. Passive is just applying to the surface of the scaffold. Additional agents, as describe above, can be further combined with the cells and scaffold.

Another aspect of this invention provides a method for generating pilosebaceous units in a physiological plane in a mammal in need thereof, comprising implanting the composition of this invention into the dermal layer of the mammal under conditions that favor implantation of the composition into the dermis of the mammal. In one aspect, the resulted concentration of cells in the scaffold is from about 800,000 cells/mm³ to about 1,500,000 cells/mm³. In some aspects, the concentration of cells in the scaffold is equal to or greater than about 10,000 cells/mm³, or alternatively is equal to or greater than about 50,000 cells/mm³, alternatively is equal to or greater than about 100,000 cells/mm³, alternatively is equal to or greater than about 200,000 cells/mm³, alternatively is equal to or greater than about 300,000 cells/mm³, alternatively is equal to or greater than about 400,000 cells/mm³, alternatively is equal to or greater than about 500,000 cells/mm³, alternatively is equal to or greater than about 600,000 cells/mm³, alternatively is equal to or greater than about 700,000 cells/mm³, alternatively is equal to or greater than about 800,000 cells/mm³, alternatively is equal to or greater than about 900,000 cells/mm³, or alternatively is equal to or greater than about 1,000,000 cells/mm³. In some aspects, the concentration of cells in the scaffold is equal to or less than about 800,000 cells/mm³, or alternatively is equal to or less than about 900,000 cells/mm³, or alternatively is equal to or less than about 1,000,000 cells/mm³, or alternatively is equal to or less than about 1,100,000 cells/mm³, or alternatively is equal to or less than about 1,200,000 cells/mm³, or alternatively is equal to or less than about 1,300,000 cells/mm³, or alternatively is equal to or less than about 1,400,000 cells/mm³, or alternatively is equal to or less than about 1,500,000 cells/mm³, or alternatively is equal to or less than about 1,600,000 cells/mm³, or alternatively is equal to or less than about 1,700,000 cells/mm³, or alternatively is equal to or less than about 1,800,000 cells/mm³, or alternatively is equal to or less than about 1,900,000 cells/mm³, or alternatively is equal to or less than about 2,000,000 cells/mm³, or alternatively is equal to or less than about 5,000,000 or alternatively is equal to or less than about 10,000,000 cells/mm³. In one aspect, the skin precursor cells comprise epidermal and dermal precursor cells. In some embodiments, the ratio of epidermal to dermal precursor cells is about 2:1, or alternatively is about 1:1, or alternatively is about 1:2, or alternatively is about 1:3, or alternatively is about 1:4, or alternatively is about 1:5, or alternatively is about 1:6, or alternatively is about 1:7, or alternatively is about 1:8, or alternatively is about 1:9, or alternatively is about 1:10, or alternatively is about 1:12, or alternatively is about 1:15, or alternatively is about 1:20 or alternatively is about 1:50. In some embodiments, the conditions that favor implantation of the composition into the dermis of the mammal comprise, or alternatively consist essentially of, or yet further consists of applying suitable pressure to maintain contact between the composition and the muscle or subcutaneous fat of the mammal for at least 3 days. In some embodiments, the conditions that favor implantation of the composition into the dermis of the mammal comprise applying a dressing on top of the composition. In some embodiments, the dermal layer of the mammal was pretreated with an effective amount of an agent that inhibits Bone Morphogenic Protein (BMP) signaling. In one aspect of the embodiments, the agent is selected from the group consisting of dorsomorphin, noggin, chordin, gremlin, sclerostin and follistatin and combinations thereof.

In a further aspect, the cells further contain a detectable label that can be used to monitor the growth and differentiation of the cells in the subject.

In another aspect, optical methods can be used to monitor the growth and differentiation of the cells. Stem cells can be transduced using the lentivirus under the control of a constitutively active ubiquitin promoter driving the expression of luciferase. Animals can be anaesthetized, injected with D-luciferin and bioluminescence imaging can be performed in vivo using a Xenogen IVIS 200 System cooled CCD camera (Cheng et al. (2006) Bioconjug. Chem. 17:662-669; Love et al. (2007) J Nucl. Med. 48(12):2011-20). Bioluminescence imaging can be used to check if these stem cells stay in the skin, close to where they were injected, or if they become diffuse, invasive, or distributed all over the body. It can also be checked if these cell products stay organized or start to become disorganized. Although the resolution of luciferase imaging is 1-2 mmm in vivo, the technique is sensitive, less costly, and has a higher temporal resolution (milliseconds)(Miller (2004) Adv. Drug Deliv. Rev. 56(12):1811-24) than other techniques. For in vivo detection of organization, GFP can be used. ES cells can be made to express GFP constitutively. Animals can also be engineered to express GFP constitutively. Transplantation of these cells onto a GFP negative host will allow one to visualize the organization of GFP positive cells using fluorescent microscopy (e.g. Leica Z16 APO fluorescent microscope). While the resolution of fluorescent imaging is much better, the light penetration is not good.

In a further aspect, micro PET/CT can be used for long-term tracking. To create a positron emission tomography (PET) reporter gene system, stem cells can be transduced with Herpes Simplex virus type-1 thymidine kinase (HSV-TK1) under the control of the constitutively active ubiquitin promoter. PET scans of stem cells preloaded with ¹⁸F-FDG or ⁶⁴Cu-PTSM provide imaging over only a period of days, because of the loss of signal from radioactive decay. HSV-TK1 high affinity PET radiotracer 9-[4-[¹⁸F]fluoro-3-(hydroxymethyl)butyl]guanine ([¹⁸F]FHBG) is also appropriate. PET scans with [¹⁸F]FHBG allows the flexibility to monitor stem cells over a span of several weeks through multiple time points. In addition, images can be acquired in conjunction with bioluminescence imaging Concorde Microsystems microPET R4 can be used, immediately followed by a CT scan using the Siemens Inveon microCT to produce co-registered PET/CT images. The PET data can then be reconstructed using the Maximum a Posteriori image (MAP) reconstruction, to provide higher spatial resolution PET images. The PET information acquired on the microPET can be co-registered with the CT data to provide the combination of stem cell location (PET data) layered on an anatomical reference image (CT data).

In yet another aspect, ultrasound can be used for tracking the cells. For ultrasound imaging, the skin is first shaved (not plucked) to avoid any damage to the hair follicles. The skin is then covered with aquasonic gel to facilitate contact of the ultrasound probe. Images are videotaped to produce real time movies of the skin. The overall architecture can be visualized.

### Therapeutic and Diagnostic Utilities

In one aspect, the invention provides a method for generating pilosebaceous units in a physiological plane in a mammal in need thereof, comprising, or alternatively consisting essentially of, or yet further consisting of implanting the composition of the invention into the dermal layer of the mammal under conditions that favor implantation of the composition into the dermis of the mammal. In one aspect, the composition replaces the entire skin within the area. In another aspect, the epidermis and all of the dermis are replaced by the composition. In yet another aspect, the epidermis and part of the dermis are replaced by the composition. One can determine when the method has been accomplished by noting the growth of hair or formation of pilosebaceous units in a topical plane in the mammal.

In another aspect, the invention provides a method for preparing pilosebaceous units in a physiological plane, comprising admixing skin precursor cells and a medium, wherein the concentration of skin precursor cells present in the medium is greater than about 1×10⁷ cells per milliliter of medium. In some embodiments, the concentration of skin precursor cells present in the medium is less than about 1×10⁸ cells per milliliter of medium. Yet in some embodiments, the concentration of skin precursor cells present in the medium is from about 2×10⁷ cells per milliliter of medium, or alternatively about 3×10⁷ cells per milliliter of medium, or alternatively about 4×10⁷ cells per milliliter of medium, or alternatively about 5×10⁷ cells per milliliter of medium to about 6×10⁷ cells per milliliter of medium, or alternatively about 7×10⁷ cells per milliliter of medium, about 8×10⁷ cells per milliliter of medium, or alternatively about 9×10⁷ cells per milliliter of medium, or alternatively about 1×10⁸ cells per milliliter of medium.

The skin precursor cells in the medium are comprised, or alternatively consisting essentially of, or yet further consisting of dermal precursor cells and epidermal precursor cells. In some embodiments, the ratio of epidermal to dermal precursor cells is about 2:1, or alternatively about 1:1, or alternatively about 1:2, or alternatively about 1:3, or alternatively about 1:4, or alternatively about 1:5, or alternatively about 1:6, or alternatively about 1:7, or alternatively about 1:8, or alternatively about 1:9, or alternatively about 1:10, or alternatively about 1:12, or alternatively about 1:15, or alternatively about 1:20 or alternatively about 1:50. In some aspects, the composition can further comprise, or alternatively consist essentially of, or yet further consist of, an effective amount of a suitable carrier and/or a growth or differentiation factor. In one aspect, the factor is one or more of noggin, chordin, gremlin, sclerostin or follistatin. In another aspect, the factor is one or more of Platelet Derived Growth Factor (PDGF), Vascular Endothelial Growth Factor (VEGF), Epithelial Growth Factor (EGF), TGF-β, Fibroblast Growth Factor (FGF), insulin, transferring or retinoid.

The skin precursor cells and the medium are admixed on any surface or within a container suitable for cell culture such as a cell culture insert as described herein. The size and shape of the surface or container is unlimited. In some embodiments, the container has a volume that is about 50 μl, or alternatively about 100 μl, or alternatively about 200 μl, or alternatively about 500 μl, or alternatively about 1 ml or more. The shape of the container is non-limiting. In some embodiments, the container is round, or alternatively square, or alternatively adopts the shape of the intended implant.

Suitable medium includes, without limitation, 1:1 DMEM/F12 with no serum. The cell slurries that form after the skin precursor cells and the mediums are mixed and allowed to settle down for about 1 to about 2 hours in an incubator set at a temperature of about 37° C. before grafting onto the host. The cell slurry is then can then be grafted onto the host. In transplantation, the cell slurry can be placed under a piece of epithelial sheet. The typical size is about 1.5 cm² although any range of up to about 2.5 mm in diameter or about 5 by 40 mm will also sufice.

Suitable membranes for the slurry include, without limitation, Integra™ and Falcon™ tissue culture insert. The Integra™ matrix is commercially available. The culture insert membrane (polyethylene terephthalate PET) is also commercially available from,e e.g., BD Falcon, San Jose, Calif.

In some embodiments, the method further comprises, or alternatively consists essentially of, or yet further consists of, the step of overlaying an epithelial sheet on the admixed dermal precursor cells and the medium. The pilosebaceous units prepared by the method of the invention can be used to treat a condition in a mammalian subject in need of, which condition comprises hair loss or insufficient hair growth. In one aspect, the condition is alopecia. In another aspect, the condition is wound healing.

Mammals that may be suitably treated by this method include, but are not limited to those described as “subjects” herein. It is apparent to those skilled in the art that the cell source for therapeutic use should match or closely match the species into which cells and matrix are implanted. For example, when the method is practiced a human patient, the cell source should be human as well. However, when the purpose of the invention is to screen agents that can modulate the formation of pilosebaceous units in a topical plane, it is not necessary that the source of cells be identical to the subject being treated. It is conceivable that human cell sources may be implanted into mice (nude mice as shown below) and then agents are contacted with the implant either by incorporation into the matrix or alternatively by subsequent administration to the implanted cells and growth is monitored. The test agents can be compared to known agents that modulate hair growth, for example noggin or Minoxidol™ to determine if they are candidate leads for further development. This is a fast and simple clinically relevant animal model for high-throughput screening of various test agents.

In yet another aspect, the conditions that favor implantation of the composition or the scaffold into the dermis of the mammal comprises suitable pressure to maintain contact between the composition and the muscle or subcutaneous fat of the mammal for at least 3 days or alternatively at least 5 days, or yet further at least 7 days. In one aspect, pressure is maintained by covering the implant with a silicone covering for an effective amount of time.

In yet another aspect, the composition of this invention can be used to treat a condition in a mammalian subject in need of, which condition comprises hair loss or insufficient hair growth. In one aspect, the condition is alopecia. In another aspect, the condition is wound healing.

The agents and compositions of the present invention can be used in the manufacture of medicaments and for the treatment of humans and other animals as described and exemplified herein.

This invention also provides a non-human animal model to screen for agents that modulate the growth of hair in a physiological plane comprising, or alternatively consisting essentially of, or yet further consisting of a suitable subject having implanted into the tissue of the subject an effective amount of the cell and scaffold matrix as variously described above. The agent to be screened can be added to the scaffold/cell composition or alternatively, subsequently applied to the area of an animal or human that received the implant. The growth of hair and/or formation of pilosebaceous units is monitored and alternatively can be compared to a second animal receiving the same implant without the test agent or yet further or alternatively a third animal receiving a known agent such as noggin that modulates hair growth. Agents can either augment (support) or impede hair growth or the formation of pilosebaceous units. Alternatively, they may have substantially no therapeutic impact. However, agents that do modulate can be selected for further research and clinical development.

The agents, compositions and methods of the present invention in any of the above embodiments can be used in the manufacture of medicaments and for the treatment of humans and other animals by administration in accordance with conventional procedures, such as an active ingredient in pharmaceutical compositions.

### Kits

An aspect of the invention provides a kit for performing at least one therapeutic or diagnostic method of this invention comprising, or alternatively consisting essentially, or yet further consisting of an effective amount of a suitable biocompatible matrix and instructions for use which may include methods to isolate the precursor cells. In some embodiments, the pharmaceutically acceptable carrier in the kits is suitable for topical administration of the agent. In some embodiments, the pharmaceutically acceptable carrier further comprises a penetration or permeation enhancer.

Also provided are kits for administration of the compounds for treatment of disorders as described herein. Kits may further comprise suitable packaging and/or instructions for use of the cells and scaffold. Kits may also comprise a means for the delivery of the at least one agonist or antagonist and instructions for administration. Alternatively, the kit provides the compound and reagents to prepare a composition for administration. The composition can be in a dry or lyophilized form or in a solution, particularly a sterile solution. When the composition is in a dry form, the reagent may comprise a pharmaceutically acceptable diluent for preparing a liquid formulation. The kit may contain a device for administration or for dispensing the compositions, including, but not limited to, syringe, pipette, transdermal patch and/or microneedle.

The kits may include other therapeutic compounds for use in conjunction with the compounds described herein. These compounds can be provided in a separate form or mixed with the compounds of the present invention.

The kits will include appropriate instructions for preparation and administration of the composition, side effects of the compositions, and any other relevant information. The instructions can be in any suitable format, including, but not limited to, printed matter, videotape, computer readable disk, or optical disc.

In another aspect of the invention, kits for treating an individual who suffers from or is susceptible to the conditions described herein are provided, comprising a container comprising a dosage amount of a composition as disclosed herein, and instructions for use. The container can be any of those known in the art and appropriate for storage and delivery.

Kits may also be provided that contain sufficient dosages of the effective composition or compound to provide effective treatment for an individual for an extended period, such as a week, 2 weeks, 3, weeks, 4 weeks, 6 weeks, or 8 weeks or more.

The following examples are provide to illustrate select embodiments of the invention as disclosed and claimed herein.

### EXPERIMENTAL EXAMPLES

### Example 1

### A Simplified Planar Hair Forming Protocol for High Throughput Assay Clinical Use

This example shows a method in which a large number of pilosebaceous units, including hair, are able to be grown from a dissociated cell suspension in vivo. While methods for hair generation have been described in the past, this invention is an improvement over prior art methods by making the process more user friendly for the clinician as well as more comfortable for the patient or animal. Additionally, by seeding these cells in a scaffold-like scaffold, it was unexpectedly shown that the cells will reorganize themselves in a way such that proper orientation of hair growth is obtained in a cosmetically acceptable manner. This protocol can be useful for high throughput screening of molecules or drugs important for hair formation. It may also have future clinical benefits for hair regeneration after severe wound injury and the treatment of alopecia.

### Material and Methods:

Cell Isolation: Multipotential skin precursor cells are currently obtained from neonatal mice using techniques from previously published work (see e.g. Lichit et al. (1993) J. Invest. Dermatol. 101(1 Suppl):124S-129S). Briefly, neonatal mice are harvested shortly after birth (within the first 24 hours) and euthanized. The truncal skin is then dissected off with sharp forceps. Epidermis and dermis are separated by floating in cold 0.25% trypsin overnight. Epidermal cells are then dissociated into a single cell suspension by cutting into fine pieces and manual tituration with a pipet. The dermal cells are individually dissociated using warm 0.35% collagenase for 30 minutes at 37° C. and then manual titration. The collagenase and trypsin activities are stopped by washing cells in either trypsin inhibitor or medium containing a 10% fetal bovine serum. The cells are passed through a 100 μm cell strainer to ensure single cell suspension and exclude cells of the stratum corneum. Both sets of cells are then recombined in a ratio of 1 epidermal to 5 dermal cells and washed again in Dulbecco's Modified Eagle Medium (DMEM): Nutrient Mixture F-12 (F12) (1:1). The cells are finally re-suspended into 150-200 μl of DMEM:F12 (1:1) as a slurry.

Seeding into Biocompatible Scaffold: Integra Bilayer Matrix Wound Dressing (Integra Life Sciences Corporation, Plansboro, N.J.) is used in this example. Treatment of the collagen matrix begins by rinsing several times with normal, serum free medium. Once this is done, the matrix is cut into the appropriate size and dried with sterile, non-stick gauze. The slurry of recombined cells are then placed onto the undersurface of the dry collagen matrix using a pipet. For this experiment, approximately 12 million epidermal cells and 60 million dermal cells in 200 μl of serum free medium for each 1.5 cm² (1 mm thick) piece of matrix. However, prior experiments show that hairs can be grown without the commercially available Integra matrix, rather re-suspending the cells, after washing in DMEM:F12 (1:1), in type I collagen or without any collagen and pipeted the slurry onto a silicone cell culture insert. In the case of the matrix purchased from Integra, cells are allowed to soak into the matrix for 1-2 hours in a 37° C. incubator. Similarly, cells are incubated on the silicone cell culture insert to allow for a gel like scaffold to form and excess liquid to dry.

Grafting Procedure: The in vivo graft bed is prepared using sterile technique. A full thickness piece of skin, approximately the size of the collagen matrix, is excised. Once bleeding has been controlled, the collagen matrix or silicone cell insert, with the cells seeded inside or on top, is grafted by suture ligation such that the cells are against the wound bed with a silicone protective layer level with the epidermis. Sterile dressing are applied in order to provide constant pressure against the graft to the wound bed so that the graft has the best chance of capillary formation and being incorporated as part of the host's skin. Dressings are then taken down for inspection of the wound on days 6-8 post graft. The sutures are removed and the protective silicone layer that had been on the matrix is easily peeled off at this time. No special care of the animal is needed once dressings have been taken down. Hair follicles can be seen by the naked eye on the surface of the animal's skin as early as 12-14 days post graft.

### Results:

It is now possible to obtain skin precursor cells, or multipotential skin stem cells, from different sources. Dermal and epidermal precursor cells have to be recombined to form skin appendages. In general, it is known that skin appendages, however disorganized, can form resulting from an epidermal-dermal interaction. Mammalian, newborn epidermal and dermal skin cells were isolated from the stratum corneum of the tissue and placed them into a scaffold-like matrix that is easily grafted and allows the cells to reorganize in a proper topological plane. This method allows cells to self-organize in a scaffold so that a large number of hair follicles can be generated easily and distributed in a plane with a cosmetically acceptable arrangement. This method is highly reproducibly that cells can be easily grafted with the help of a scaffold-like matrix to allow cells to reorganize and grow new hair in a cosmetically acceptable fashion. Hairs have been allowed to grow up to 3 months, giving evidence that the hair is permanent and able to cycle normally.

Different cell ratios between the epidermal and dermal populations have been tested to identify an ideal cell ratio or total cell number to use in this assay (Table 1). The types of skin precursor cells used, types of biocompatible scaffold may also have impact on formation of hair follicles in a physiological plane. As shown in Table 1, a ratio of epidermal and dermal precursor cells between 1:5 and 1:10 gave better results than that of 1:2. A combination of aged epidermal cells and newborn dermal cells, or a combination of newborn epidermal cells and aged dermal cells did not give rise to good hair growth. However, it has been noted that a replacement of newborn epidermal cells with aged epidermal cells had a lesser effect than a replacement of newborn dermal cells with aged dermal cells. GFP positive precursor cells also showed good hair growth. A combination GFP positive precursor cells and whole skin (WT) cells led to fair hair growth as well. While the use of Integra Matrix produced good hair growth, use of other scaffold such as type I collagen resulted in mostly good hair growth too.

TABLE 1
Factors affecting PHP (Physiological Hair Plane)

| | Total | | | |
|---|---|---|---|---|
| | Number of | Good Hair | Fair Hair | |
| | Animals | Growth | Growth | None |
| Whole skin (WT) | 7 | 5 | 2 | 0 |
| Ratio 1:2 | 2 | 0 | 2 | 0 |
| Ratio 1:5 | 2 | 2 | 0 | 0 |
| Ratio 1:10 | 2 | 2 | 0 | 0 |
| Aged Epidermal:New- | 2 | 0 | 0 | 2 |
| born Dermal | | | | |
| Aged Dermal:Newborn | 2 | 0 | 0 | 2 |
| Epidermal | | | | |
| GFP (Green Fluorescent | 1 | 1 | 0 | 0 |
| Protein) | | | | |
| GFP:WT | 2 | 0 | 2 | 0 |
| Non-Integra | 4 | 3 | 1 | 0 |
| Lentivirus Transduced | 1 | 0 | 0 | 1 |
| Cells | | | | |

### Discussion:

With previous methods, the formed hairs are randomly positioned, forming a hair cyst, rather than hair. While the old procedures are useful for science assays, disorganized or inward growth of hair follicles is not useful for practical use. Because hair follicles grow in an inward direction with the previous method, they are unable to cycle and grow normally. This invention provides a procedure that allows precursor cells to self-organize in order to generate a large number of new hair follicles which are arranged in a plane with a cosmetically acceptable appearance. The formed hairs are able to cycle through the normal hair cycles of regeneration. This procedure can be performed efficiently, reproducibly and on a large scale so that clinical applications can be envisioned. Furthermore, the hair grows in a topologically proper environment such that there is room for cycling and hairs to fall out and regrow. This technology is useful to form skin with hairs on patients suffering from severe injuries such as burn or alopecia patients.

### Example 2

### Prepare Skin Stem Cells and Their Environment for Delivery

Given one million skin cells capable of forming hairs, 10 big hairs or 1000 small hairs can be formed. Big hairs are generally the preferred outcome. The regulatory patterning of this process has been studied by the inventors (Maini et al. (2006) Science 314(5804):1397-1398). In vitro studies showed that Turing reaction-diffusion plays a critical role in periodic patterning a homogenous population of stem cells (Jung et al. (1998) Dev Biol. 196(1):11-23; Jiang et al. (1999) Development 126(22):4997-5009). This suggests that the system can self-organize and cells respond to their local environment to assemble organs with a specific architecture based on cells ability to use their intrinsic (e.g. growth factor receptors, adhesion molecules) and extrinsic (e.g. growth factors, extracellular matrix molecules, etc.) properties. Before explant culture, dissociated multi-potential skin precusor cells are pre-plated at high cell density (15 μl of a 2×10⁷ cells/ml suspension) on a cell culture insert (Falcon). This is a critical step which allows cells to sort themselves out and lay down essential extracellular matrix molecules. An epithelial sheet is then overlaid. By varying the ratio of Turing activator/inhibitor in this micro-environment, the size of feather buds are modulated. By varying the number of competent dermal cells, the number of feather buds are altered, but the bud size remains constant.

Several growth factor pathways are shown to be involved in hair follicle formation (Millar (2002) J. Invest. Dermatol. 118(2):216-25.) by acting as activators (Wnt/beta catenin FGF pathways) or inhibitors (BMP pathway) of skin appendage formation. Small or large molecules exist that regulate these pathways thereby facilitating hair formation.

The regenerative behavior of hair follicle population in living mouse has been well studied by the inventors. The results showed that hair stem cell activity is not only regulated by its immediate micro-environment but also by the macro-environment, meaning the environment outside of the follicle which includes adjacent dermis and body hormone conditions (Plikus et al. (2008) Nature 451(7176):340-344). BMP signaling confers refractory (high BMP) or competent (low BMP) status to adjacent hair follicles. This novel finding has a broader significance. It implies that when one delivers stem cells to a location, one should check and choose the site that is in the more favorable competent states. Alternatively, one could prepare the transplantation site by treatment with some molecules, thus making it a more favorable environment. The target skin of the treatment can be pretreated with noggin or various small molecules that inhibit BMP pathway activity. For example, dorsomorphin is a potent small molecule BMP antagonist (Hao et al. (2008) PLoS ONE 3(8):e2904, Yu et al. (2008) Nat. Chem. Biol. 4(1):33-41). Dorsomorphin was reported to selectively inhibit the BMP receptors, type I: ALK2, ALK3 and ALK6 and thus “blocks BMP-mediated SMAD1/5/8 phosphorylation”. Dorsomorphin has preferential specificity toward inhibiting BMP vs. TGF-beta and activin signaling. In published reports, Dorsomorphin is characterized by low toxicity. Dorsomorphin is currently commercially available from several vendors. Dorsomorphin can be delivered into skin to lower macro-environmental BMP signaling and create favorable conditions for hair growth to occur.

### Results

The size of a hair is determined by the size of its dermal papilla, which in turn is determined by the size of the dermal condensation during development. Reaction diffusion mechanism can set self-organization in motion, but the outcome (number and size of dermal condensations) is modulated by the initial parameters (size of field, ratio of activator/inhibitor activity, number of activator and inhibitor receptors, etc.) (Jiang et al. (2004) Int. J. Dev. Biol. 48(2-3):117-135). The results are judged in whole mount and in histological sections. The number and size of hair follicles are quantified for each experiment and the results of at least 5 specimens are averaged for each experimental reagent and compared with control with are treated with vehicles. Alternatively, the formation of bigger dermal condensations are enhanced by pre-shaking cells in an environmental shaker to facilitate the formation and stabilization of bigger cell aggregates.

### Example 3

### A Simplified Planar Hair Forming Protocol for High Throughput Assay Clinical Use

In an extension of the experiment described in Example 1, Applicant provides the following Example 3.

### Material and Methods:

Cell Isolation: Multipotential skin precursor cells are currently obtained from neonatal mice using techniques from previously published work. Briefly, neonatal mice are harvested shortly after birth (within the first 24 hours) and euthanized. The truncal skin is then dissected off with sharp forceps. Epidermis and dermis are separated by floating in cold 0.25% trypsin overnight. Epidermal cells are then dissociated into a cell suspension by cutting into fine pieces and manual tituration with a serological pipet. Single epithelial cells are separated through a 70 μm cell strainer to exclude cells of the stratum corneum. The dermal cells are individually dissociated using warm 0.35% collagenase for 40-50 minutes at 37° C. DNase I is then added for 5 minutes at RT before manual tituration with a serological pipet. The collagenase and trypsin activities are stopped by washing cells in either trypsin inhibitor or medium containing a 10% fetal bovine serum. The cells are passed through a 40 μm cell strainer to ensure single cell suspension and exclude as many of the pre-formed hair follicles as possible. Both sets of cells are then recombined in a ratio of 1 epidermal to 5-10 dermal cells and washed again DMEM:F12 (1:1). The cells are finally resuspended into 150-200 μl of DMEM:F12 (1:1) as a slurry.

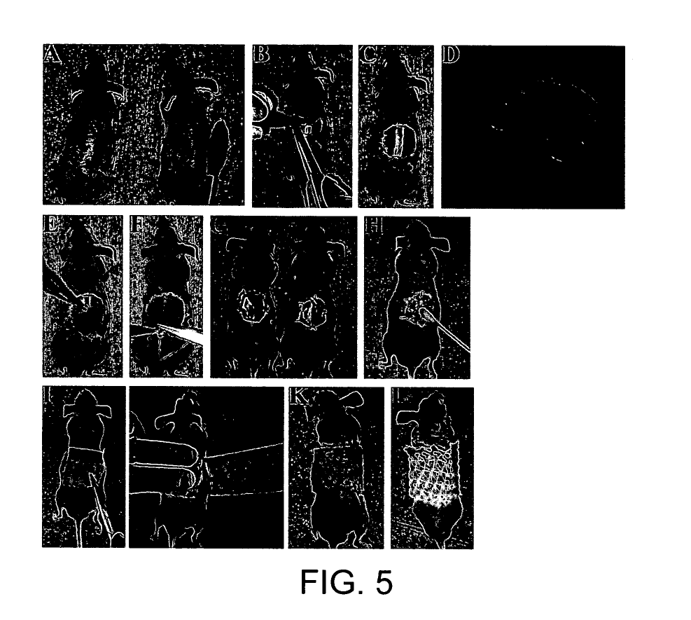

Seeding into Matrix: Integra is used in this example. Treatment of the collagen matrix begins by rinsing several times with normal, serum free medium. Once this is done, the matrix is cut into the appropriate size and dried with sterile, non-stick gauze. The slurry of recombined cells is then placed onto the undersurface of the dry collagen matrix using a pipet. Currently approximately 12 million epidermal cells and 60 million dermal cells are used in 200 μl of serum free medium for each 1.5 cm² (1 mm thick) piece of matrix. Hairs have also been successfully grown without the Integra matrix, rather resuspending the cells, after washing, in the smallest amount of medium possible and pipeted the slurry onto a silicone cell culture insert. In the case of Integra, cells are allowed to soak into the matrix for 1-2 hours in a 37° C. incubator. Similarly, cells are incubated on the silicone cell culture insert to allow for a gel like scaffold to form and excess liquid to dry. (FIG. 5D).

Grafting Procedure: (FIG. 5) The in vivo graft bed is prepared using sterile technique. A full thickness piece of skin, approximately the size of the collagen matrix, is excised. Once bleeding has been controlled, the collagen matrix or silicone cell insert, with the cells seeded inside or on top, is grafted by suture ligation such that the cells are against the wound bed with a silicone protective layer level with the epidermis. Sterile dressing are applied in order to provide constant pressure against the graft to the wound bed so that the graft has the best chance of capillary formation and being incorporated as part of the host's skin. Dressings are then taken down for inspection of the wound on days 7-9 post graft. The sutures are removed and the protective silicone layer that had been on the matrix is easily peeled off at this time. No special care of the animal is needed once dressings have been taken down. Hair follicles can be seen by the naked eye on the surface of the animal's skin as early as 11-15 days post graft.

### Results:

It is now possible to obtain skin precursor cells, or multi-potential skin stem cells, from different sources. Dermal and epidermal precursor cells have to be recombined to form skin appendages. In general, it is known that skin appendages, however disorganized, can form resulting from an epidermal-dermal interaction.

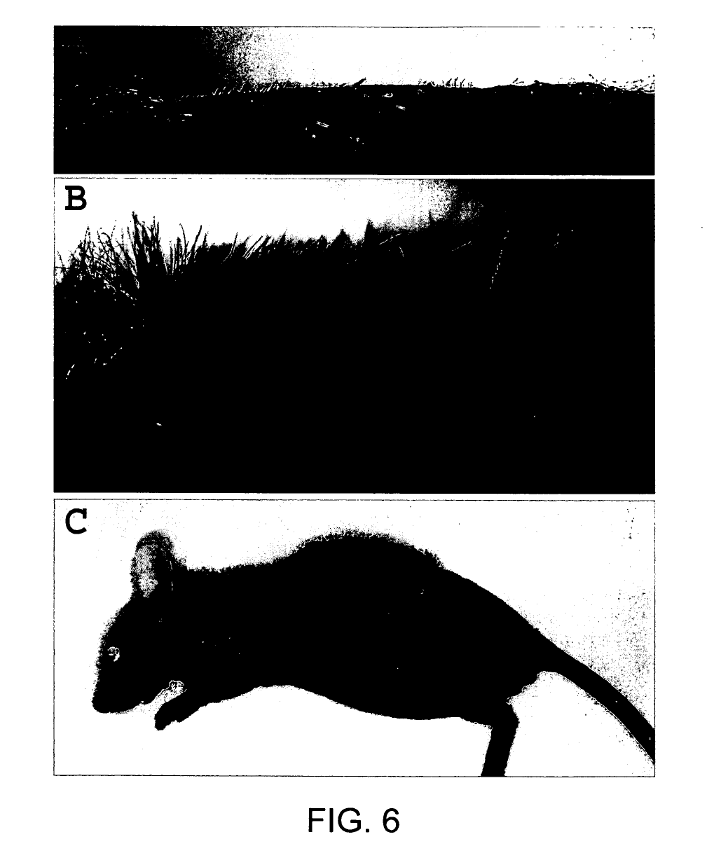

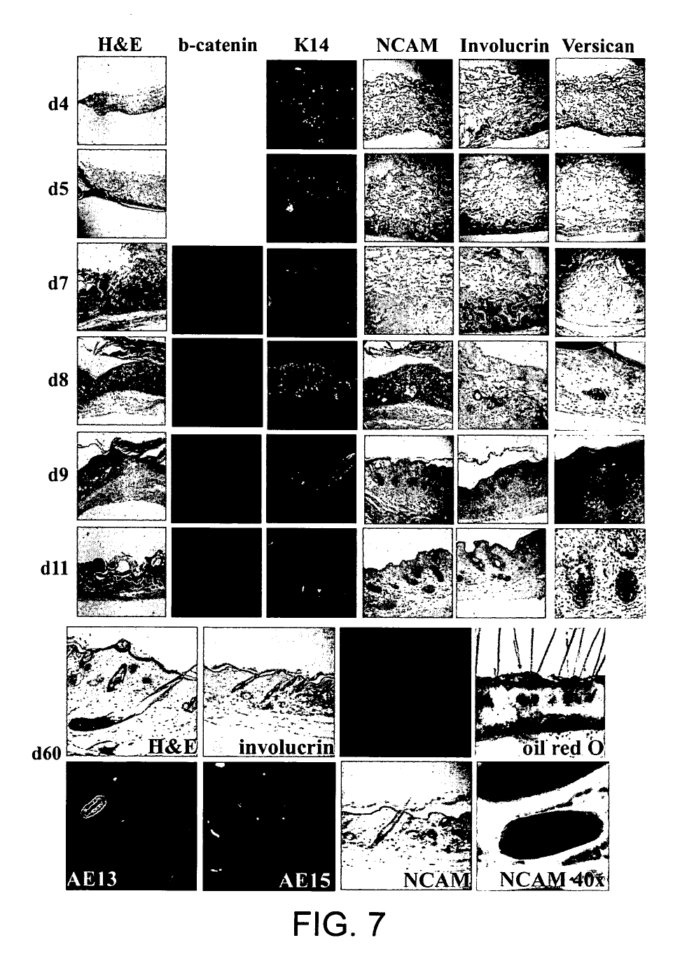

Topology: Newborn mouse epidermal and dermal skin cells have been taken and placed them into a scaffold-like matrix that is easily grafted and allows the cells to reorganize in a proper topological plane. The results are easily reproducible and mimic the natural in vivo situation. (FIG. 6). Histologic data on serial sections of post operative days shown that cells, particularly epithelial in origin, rise from the base (where they are seeded) through the Integra matrix to the level of the air surface level (FIG. 7). The cells organize themselves into epidermal and dermal layers and then further into pilosebaceous units. No hair follicles growing on the underside of the matrix have been found unless the matrix is overloaded with cells and form a cyst, similar to the patch assay, distant from the grafted area. This suggests that there must be a homing mechanism in which epithelial cells know to organize themselves around and on top of dermal cells. The current discovery is to allow cells to self-organize in a scaffold so that a large number of hair follicles can be generated easily and distributed in a plane with a cosmetically acceptable arrangement.

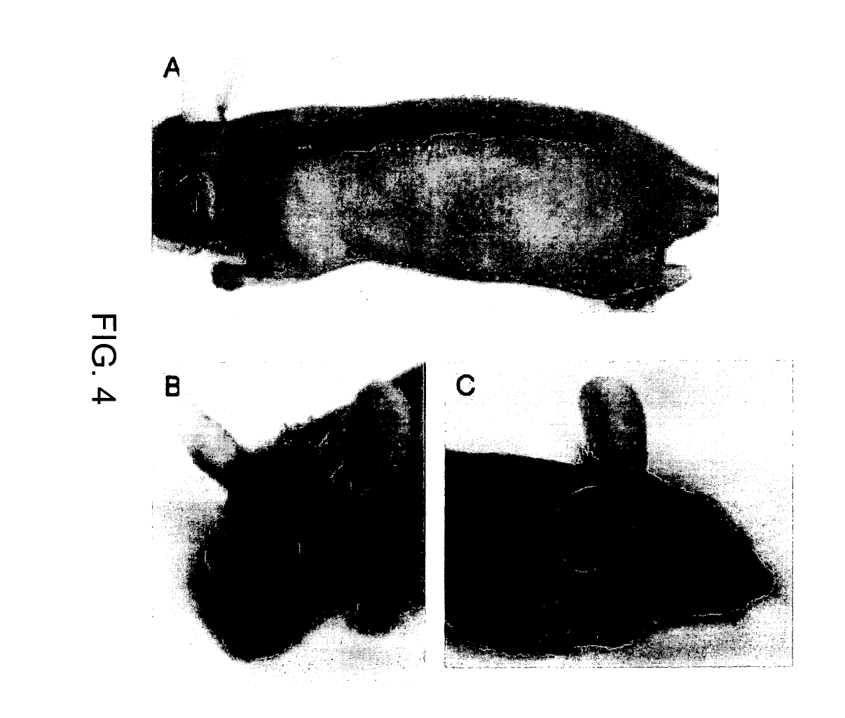

Arrangement/shaping: Because the matrix is reasonably stiff, it holds cells within the sponge-like structure and can be shaped as needed. This can conceivably be made clinically useful in the reconstructive procedures of hair replacement of particularly shaped regions of hair growth (i.e. Eyebrow) (FIG. 4). Regions of hair replacement can be made as a large as 30% of the patients body (depending of tolerance) or as small as the operator is able to make the matrix. Grafts as small as 0.5 cm² have been made.

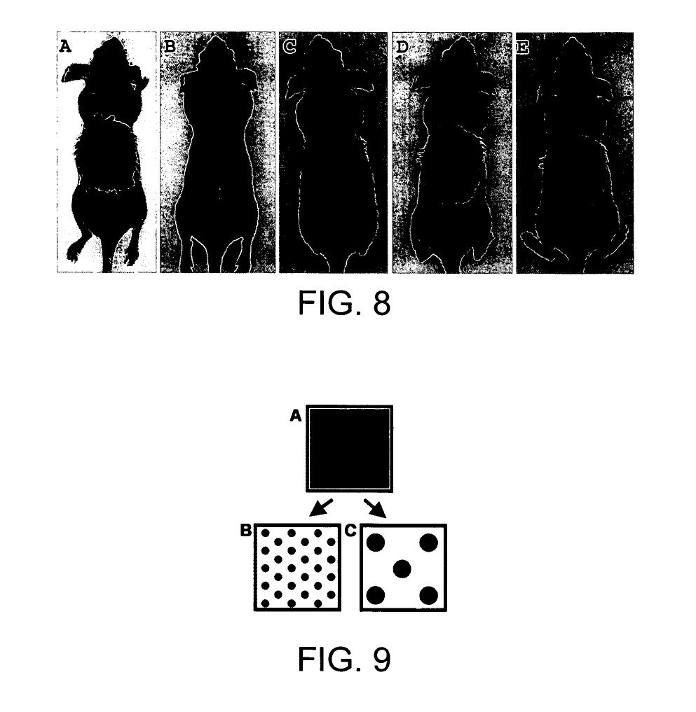

Organization: It is shown with high reproducibility that cells can be easily grafted with the help of a scaffold-like matrix to allow cells to reorganize and grow new hair in a cosmetically acceptable fashion. Not only do these hairs appear cosmetically acceptable, the histologic sections show that these hair recreate all the normal layers of real hair, glands and skin (FIG. 7). Hairs have been observed to grow up to 18 months, giving evidence that the hair is permanent. Further, it has been tested the hair by shaving and plucking to show regeneration and normal cycling (FIG. 8). Additionally, different cell ratios, between the epidermal and dermal populations, have been tested and show that with the method of the current invention, a ratio of epidermal:dermal cells of about 1:5-10 is optimal.

Discussion: With previous methods, the formed hairs are randomly positioned, forming a hair cyst, rather than hair. While the old procedures are useful for science assays, disorganized or inward growth of hair follicles is not useful for practical use. Because hair follicles grow in an inward direction with the previous method, they are unable to cycle and grow normally. The current invention provides a procedure that allows precursor cells to self-organize in order to generate a large number of new hair follicles. To Applicant's knowledge, the method described most closely mimics arranged hairs in an in vivo manner. The hair grown allows for large portions of hair bearing skin to be cosmetically acceptable in appearance and normal function. The formed hairs are able to cycle through the normal hair cycles of regeneration.

### Example 4

### A Simplified Planar Hair Forming Procedure

In an extension of the experiments described in Examples 1 and 3, Applicant provides the following Example 4.

The present invention provides a simplified planar hair forming procedure. The protocol is easy to use, easier to endure for animals or patients, and can be standardized to show reliable outcome in a timely manner. This protocol is useful for the large scale assessment of factors that can modulate the hair formation ability of cells and may lead to future clinical benefits for burn wounds and alopecia treatment.

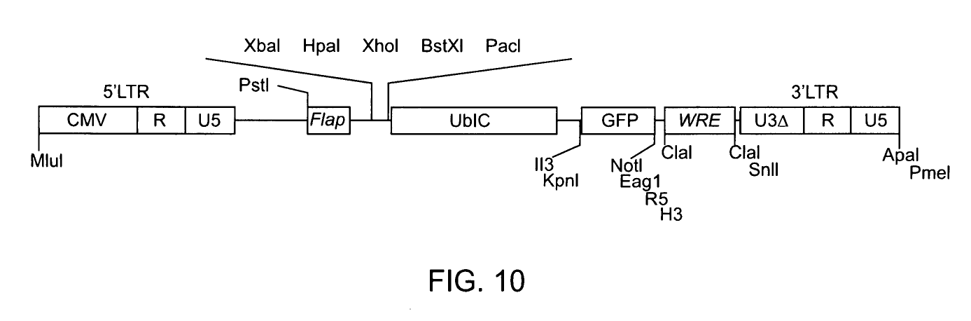

The basic protocol of the invented procedure in illustrated in FIG. 5. Characterization of the reconstituted skin is shown in FIG. 7. FIG. 8 shows physiological molting and regeneration of these hairs on the reconstituted skin. FIG. 9 is the schematic summary of different ways of patterning skim stem cells. FIG. 10 shows the feasible way of reprogramming and the use of lentivirus in this assay.

Cell Isolation: Multipotential skin precursor cells were obtained from neonatal mice using techniques from previously published work. Briefly, neonatal mice were harvested shortly after birth (within the first 24 hours) and euthanized. The trunk skin was then isolated with sharp forceps. Epidermis and dermis were separated by floating in cold 0.25% trypsin overnight. Epidermal cells were then dissociated into a cell suspension by cutting into fine pieces and manual titration with a serological pipet. Single epithelial cells were separated through a 70 μm cell strainer to exclude cells of the stratum corneum. The dermal cells were individually dissociated using warm 0.35% collagenase for 40-50 minutes at 37° C. DNase I was then added for 5 minutes at RT before manual titration with a serological pipet. The collagenase and trypsin activities were stopped by washing cells in either trypsin inhibitor or medium containing a 10% fetal bovine serum. The cells were passed through a 40 μm cell strainer to ensure single cell suspension and exclude as many of the pre-formed hair follicles as possible. Both sets of cells were then recombined in a ratio of 1 epidermal to 5-10 dermal cells and washed again in DMEM:F12 (1:1). The cells were finally resuspended into 150-200 μl of DMEM:F12 (1:1) as a slurry.

Seeding into a Matrix: When Integra matrix was used, treatment of the collagen matrix began by rinsing several times with normal, serum free medium. Once this was done, the matrix was cut into the appropriate size and dried with sterile, non-stick gauze. The slurry of recombined cells was then placed onto the under surface of the dry collagen matrix using a pipet. Currently approximately 12 million epidermal cells and 60 million dermal cells were used in 200 μl of serum free medium for each 1.5 cm² (1 mm thick) piece of matrix. Hairs without the Integra matrix have also been successfully grown, by resuspending the cells, after washing, in the smallest amount of medium possible. This cell slurry was pipeted onto a silicone cell culture insert. In the case of Integra, cells were allowed to soak into the matrix for 1-2 hours in a 37° C. incubator. Similarly, cells were incubated on the silicone cell culture insert to allow for a gel like scaffold to form and excess liquid to dry.

Grafting Procedure: (FIG. 5) The in vivo graft bed was prepared using sterile technique. A full thickness piece of skin, approximately the size of the collagen matrix, was excised. Once bleeding had been controlled, the collagen matrix or silicone cell insert, with the cells seeded inside or on top, was grafted by suture ligation such that the cells were against the wound bed with a silicone protective layer level with the epidermis. Sterile dressing was applied in order to provide constant pressure against the graft to the wound bed so that the graft had the best chance of capillary formation and being incorporated as part of the host's skin. Dressings were then taken down for inspection of the wound on days 7-9 post graft. The sutures were removed and the protective silicone layer that had been on the matrix was easily peeled off at this time. No special care of the animal was needed once dressings had been taken down. Hair follicles can be seen by the naked eye on the surface of the animal's skin as early as 11-15 days post graft.

### Example 5

### The Production of Topologically Arranged Hair Follicles Produced via the Delivery of Skin Stem Cells Using the Planar Hair Forming Procedure

Skin precursor cells can also be seeded into a specially prepared matrix which may enhance their hair-forming ability or provide better maneuverability. An acellular matrix can be prepared from E13 mouse dermis which has a high hair-inducing ability (Schedin et al. (2004) Oncogene. 23(9):1766-79; Potapova et al. (2008) Am. J. Physiol. Heart Circ. Physiol. 295(6):H2257-63). One can also try commercially available, FDA-approved Integra or Alloderm matrices. These products are already in clinical use and may accelerate the clinical application of this procedure. The procedure is flexible and allows one to shape reconstituted hair regions as may be needed for adding an eye brow or scalp hairs. The region can also be very small (<5×5 mm), making it feasible for alopecia patients.

Because the matrix is reasonably stiff, it holds cells within the sponge-like structure and can be shaped as needed. This can conceivably be made clinically useful in the reconstructive procedures of hair replacement of particularly shaped regions of hair growth (i.e. eyebrow). Further, no hair follicles have been observed to grow on the underside of the matrix.

Additionally, no teratoma formation or malignant transformation have been observed in mice more than 1 yr after the experiment, showing that the procedure is safe.

Differentiation molecular markers can be characterized. Keratins, K5 and 14 can be used to examine the outer root sheath, K6 can be used for the inner root sheath. AE 13 monoclonal antibody can be used for hair cortex and cuticle differentiation. Oil Red O and BLIMP1 can be used for sebaceous glands. NCAM, a-smooth muscle actin, versican, corin can be used for the dermal papilla. The engineered hairs can fulfill the criteria of hair follicles as defined below (Chuong et al. (2007) J. Invest. Dermatol. 127(9):2098-100). As shown in the foregoing Examples, newborn mouse skin cells gave rise to excellent hair growth. It was observed that hairs grew up to 18 months, indicating that the hairs were permanent.

### Requirements for Hair Follicles

The proximal end of the skin appendages shows a follicle configuration, with an epithelial filament coming out of the distal end of the follicle and dermal papilla sitting at the base of the follicle.
1. It has proliferating cells (TA cells) positioned proximally, and differentiating cells positioned distally, forming a proximal—distal growth mode.
2. The follicle is made of concentric layers of outer and inner root sheath, cuticle, cortex, and medulla. Although in different hair types, variations can occur with the basic design, all follicles have a distinct internal root sheath.
3. The product of a follicle, the shaft is made with a unique molecular constitution.
4. The follicle is associated with sebaceous glands.
5. A follicle has the machinery to shed an old shaft while preserving stem cells and the DP for the next cycle
6. Inherent in the follicle is the ability to regenerate a new hair organ through repeated hair cycles on the surface of the animal's skin as early as 11-15 days post graft.

### Example 6

### Pre-Arranging Skin Stem Cells and Their Environment Before Delivery

One of the major bottlenecks in patterning stem cells is, given one million cells capable of forming hairs, 10 big hairs (useful for scalp) or 1000 small hairs (useful for body skin) may grow. Methodology can be developed to modulate this process by manipulating the conditions of the micro- and macro-environments of hair stem cells.

Experimental design: To obtain best possible results, before delivery stem cells can be pre-treated and the recipient site where the stem cells will be inserted can be prepared. These steps are necessary to guide stem cells into forming the right number and size of hair primordia. Multi-potential skin stem cells, by definition, have the ability to form hairs, although they can form different numbers or sizes of hairs.

Regulating the number and size of hair primordia. In vitro study showed that Turing reaction-diffusion plays a critical role in the periodic patterning of a homogenous population of stem cells (Jung et al. (1998) Dev. Biol. 196(1):11-23; Jiang et al. (1999) Development 126(22):4997-5009). This suggests that the system can self-organize and that cells respond to their local environment to assemble organs with a specific architecture based on the cells ability to use their intrinsic (growth factor receptors, adhesion molecules) and extrinsic (growth factors, extracellular matrix molecules, etc.) properties. In the above Examples, before explant culture, dissociated multi-potential skin precursor cells were pre-plated at high cell density (15 ul of a 2×10⁷ cells/ml suspension) on a cell culture insert (Falcon). This allows cells to sort themselves out and lay down essential extracellular matrix molecules. An epithelial sheet is then overlaid. By varying the ratio of Turing activator/inhibitor in this micro-environment, one can modulate the size of feather buds. By varying the number of competent dermal cells, one can alter the number of feather buds, but the bud size remains constant.

Several growth factor pathways are involved in hair follicle formation by acting as activators (Wnt/beta-catenin FGF pathways) or inhibitors (Dkk, BMP pathway) of skin appendage formation (Millar (2002) Invest. Dermatol. 118:216-25). The effect of altering these parameters when stem cells are being patterned can be tested. The effect of small molecules such as dorsomophin which is known to inhibit the BMP pathway can also be tested (Anderson and Darshan, (2008) Nature Chem. Biol. 4(1):15-6) and may mimic the action of noggin, which makes hair germs bigger.

Preparing the environment for stem cell delivery. Recently the applicants studied the regenerative behavior of hair follicle populations in living mice over a one year-period. The results showed that hair stem cell activity is not only regulated by its immediate micro-environment but also by the macro-environment. The macro-environment is composed of the environment outside of the follicle including the adjacent dermis and circulating hormone conditions (Plikus et al. (2008) Nature 451(17):340-345). Cyclic dermal BMP expression was identified to confer refractory (high BMP) or competent (low BMP) status to the adjacent hair follicles. This novel finding has a broader significance. It suggests that when one delivers stem cells to a location, one should select the site that is in the most favorable competent state.

Alternatively, one could prepare the transplantation site by treatment with selected molecules, thus making it a more favorable environment. The mouse skin can be pre-treated with noggin or selective known small molecules that inhibit BMP pathway activity such as dorsomorphin (Hao et al. (2008) PLoS ONE, 3(8):e2904). For this purpose, the patch assay can be used, and newborn mouse skin cells can be subcutaneously injected into sites pretreated with tested or control molecules (Zheng et al. (2005) J. Invest. Dermatol. 124(5):867-76) to avoid resetting the dermis by forming big wounds. Many small-molecules are cell-permeable so there should be difficulty with delivery. In case delivery becomes an issue, small lipophilic moieties such as linear alkyl chains can be attached to enhance their cell-permeability. Alternatively, they can be encapsulated in a liposome or nanoparticle.

Results: The size of a hair is determined by the size of its dermal papilla, which in turn is determined by the size of the dermal condensation during development (Elliott et al. (1999) J. Invest. Dermatol. 113(6):873-7). The reaction-diffusion mechanism can set self-organization in motion, but the outcome (number and size of dermal condensations) is modulated by the initial parameters (size of the field, ratio of activator/inhibitor activity, number of activator and inhibitor receptors, etc.). The results can be evaluated in whole mount and in histological sections. The number and size of hair follicles can be quantified for each experiment and the results of at least 5 specimens can be averaged for each experimental reagent and compared with vehicle treated controls. It is anticipated that culturing in EVAL biomaterials can help the self-assembly of dermal papilla cells into aggregates. Alternatively, the formation of bigger dermal papilla aggregates can be enhanced by pre-shaking the dermal cells in an environmental shaker to facilitate the formation and stabilization of bigger cell aggregates. As an alternative, sites can be pretreated with a BMP suppressor (i.e., noggin, dorsomorphin) to test whether macro-environmental manipulation will enable the better growth of stem cells.

### Example 6

### Sources of Multi-Potential Skin Stem Cells and Attempt to Use Reprogramming to Make Multi-Potential Skin Stem Cells

The experiments in some of the above Examples were carried out with newborn mouse skin cells, which consisted of powerful multi-potential epidermal and dermal stem cells in the first few days after birth. Human cells in the mouse host can also be used for the procedure.

Experimental design Experiments in the foregoing Examples used combined adult or juvenile epidermal or dermal cells with newborn cells. On the other hand, cells can be reprogrammed to modulate their fate and behave more like multi-potential skin stem cells or to regain hair forming ability. Reprogramming can be achieved by environmental reprogramming (altering the cellular environment with transient culture conditions), or molecular reprogramming (with ectopic molecular expression). Human cells such as human foreskin keratinocytes can also be as the epidermal component.

Environmental reprogramming To reprogram epithelial cells, candidate cells can be incubated with micro-environments known to be capable or hair formation. Newborn mouse epidermal and dermal cells are capable of forming hairs and inducing hair follicles, respectively (Zheng et al. (2005) J. Invest. Dermatol. 124(5):867-76; Lichti et al. (2008) Nat. Protoc. 3(5):799-810). The competence of different keratinocytes to form hairs can be gauged by combining with newborn dermal cells, which is known to be capable of inducing hair formation. The ability of different dermal cells to induce new hair formation can be gauged by combining with newborn keratinocytes, which is known to be competent to respond to inducing dermal signals and form hairs. The candidates are as follows.

Adult keratinocytes. Keratinocytes can be isolated from the same patient from hair follicle enriched cells. Since outer root sheath cells, which comprise a large population, are not far from bulge stem cells in their lineage, these cells may be selected and reprogrammed easier.

Human foreskin. As cells from young children are similar to newborn mouse cells, they can exhibit stem cell characteristics and are worth evaluating. Cell lines from a commercial source can also be used.

SKP. Direct isolation of dermal papilla cells is not practical, except from vibrissa to be used as the positive control. Recently, skin derived progenitor cells (SKP) have been isolated from the dermis and shown to share many characteristics of dermal papilla cells (Fernandes et al. (2004) Nat. Cell Biol. 6(11):1082-93; Tumbar et al. (2004) Science 303(5656):359-63; Toma et al. (2005) Stem Cells 23(6):727-37). This method can be used to prepare cells that can form hairs.

Fibroblast. Dermal cells isolated from newborn human foreskin, or adult dermis, or from a dermal papilla enriched fraction can be used.

Acellular matrix. Matrix can be prepared from tissues as described (Schedin et al. (2004) Oncogene. 23(9):1766-79 and Potapova et al. (2008) Am. J. Physiol. Heart Circ. Physiol. 295(6):H2257-63). Cell candidates in an acellular matrix derived from E13 mouse skin which has strong hair inducing ability can be used. The extracellular matrix can guide fibroblasts toward hair inducing dermal papillae.

Molecular reprogramming. With the success of iPS (Yamanaka (2008) Cell Prolif. 41 Suppl. 1:51-6) and transdifferentiation of one cell type to the other

(Zhou et al. (2008) Nature 455(7213):627-32; Pearton et al. (2005) Proc. Natl. Acad. Sci. USA 102(10)3714-9), it is conceivable that scientists can reprogram cellular fates by molecular programming or re-programming. To restore the hair forming ability in adult dermal fibroblasts and keratinocytes, one can manipulate dermal fibroblasts and keratinocytes by over-expression or knockdown of different genes independently using a lenfiviral system.

Beta catenin has been shown to be pivotal for hair bulge stem cell activity (Fuchs (1999) Harvey Lect. 94:47-77), for keratinocytes candidate molecules involved in the wnt/beta-catenin pathway can be tested, such as Wnt3a, Wnt7a, beta-catenin and DKK-1.

The applicants has found that Msx2 null mice showed accelerated wound healing when a small wound is produced (Yeh et al. (2009) Wound Repair Regen. 17(5):639-48). On the other hand, Msx 2 null mice showed reduced ability to form new hair follicles in the big wound assay. Lentiral vectors expressing Msx2 can be constructed and transduced into adult keratinocytes.

For dermal cells, newborn keratinocytes have been combined with adult fibroblast cultures, 3T3 cells, and cultured dermal papilla cells. 3T3 cells gave rise to some hair formation. As reported, dermal papilla cells lose the inducing ability in culture quickly (Jahoda et al. (1984) Nature 311(5986):560-2). Since dermal papilla cells in these conditions lose cell contact, it has been suggested that there is a loss of cell-cell adhesion. NCAM has been found to be expressed in the dermal condensation and dermal papilla of feathers (Chuong and Edelman (1985) J. Cell Biol. 101(3):1009-26) and hairs (Combates et al. (1997) J. Invest. Dermatol. 109(5):672-8. Therefore NCAM can be overexpressed in the dermal cell fraction. This can be done by transduction of lentivirus-NCAM. It is expected that more NCAM per cell can lead to higher cell-cell adhesion and less cell-substrate adhesion, leading to large dermal papilla aggregate formation.

The level of these candidate genes in different stages of hair reconstitution can be measured using PCR analysis first. Their levels will also be measured in assays in which one of the components does not support hair formation (adult keratinocyte/newborn dermal cells, or newborn keratinocytes/adult fibroblasts). It is expected that the hair promoting genes will be expressed when hair reconstitution is successful but not in those when hair reconstitution does not occur.

By transducing lentiviral vectors carrying suppressor genes to the newborn keratinocytes or fibroblasts, a decrease of hair formation is anticipated. By transducing lentiviral vectors carrying the above candidate genes to the adult keratinocytes or fibroblasts, an increase in hair formation is anticipated.

### Example 7

### Preparation of Cell Slurry for Transplantation

Multi-potential dermal cells are obtained from neonatal mice by dissociating mice dermal with collagenase. The cells are then recombined with epidermis in a defined ratio in to a very small amount of medium (DMEM/F12 in the ration of 1/1). This slurry of recombined cells is adjusted to about 10-100 million cells/ml concentration.

Usually 200 μl cell suspension, in the range of 2-20 million total cell number, are used. These cells are allowed to settle (they can be simply held together as a drop by surface tension), filled in a container (round plastic well range from 5-15 mm in diameter, or can be cast in flexible plastic chamber in free form with size ranging from 5 mm² to about 500 mm² surface area) or added to the matrix (e.g., lntegra™ or Matrigel™).

The medium is 1:1 DMEM/F12 with no serum. The cell slurries that form after the skin precursor cells and the mediums are mixed can be allowed to settle down for 1-2 hours in a 37° C. incubator before grafting onto the host. The cell slurry is then grafted onto the host. In transplantation, the cell slurry is placed under a piece of membrane. This has been done for both tissue culture insert membrane or Integra. The typical size is 1.5 cm². To test the range of wound size, as small as 2.5 mm in diameter and 5×40 mm have been successfully used. Larger sizes can also be used.

Membranes can be lntegra™ and Falcon™ tissue culture insert. The Integra™ matrix is commercially available. The culture insert membrane (polyethylene terephthalate PET) is also commercially available from BD Falcon.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

1. A composition to generate pilosebaceous units in a physiological plane comprising:
a biocompatible scaffold; and
an effective amount of skin precursor cells.

2. The composition of claim 1, further comprising a dressing.

3. The composition of claim 1, wherein the concentration of cells in the scaffold is from about 800,000 cells/mm³ to about 1,500,000 cells/mm³.

4. The composition of claim 1, wherein the skin precursor cells comprise epidermal and dermal precursor cells.

5. The composition of claim 1, wherein the epidermal and dermal precursor cells are isolated or purified cells from neonatal or aged mammals.

6. The composition of claim 1, wherein the ratio of epidermal to dermal precursor cells is from about 1:5 to about 1:10.

7. The composition of claim 1, further comprising an effective amount of an agent inhibiting Bone Morphogenic Protein (BMP) signaling.

8. The composition of claim 1, wherein the agent is selected from the group consisting of dorsomorphin, noggin, chordin, gremlin, sclerostin and follistatin and combinations thereof.

9. The composition of claim 1, further comprising an effective amount of an agent promoting cell differentiation or growth.

10. The composition of claim 1, wherein the agent is selected from the group consisting of Platelet Derived Growth Factor (PDGF), Vascular Endothelial Growth Factor (VEGF), Epithelial Growth Factor (EGF), TGF-□, Fibroblast Growth Factor (FGF), insulin, transferrin, retinoid and combinations thereof.

11. The composition of claim 1, further comprising an effective amount of minoxidil, finasteride, or an agent enhancing cell growth.

12. A method for preparing pilosebaceous units in a physiological plane, comprising admixing:
an effective amount of skin precursor cells in serum-free medium; and
a biocompatible scaffold, under conditions that favor the incorporation of the cells into the biocompatible scaffold.

13. The method of claim 12, further comprising admixing a dressing.

14. The method of claim 12, wherein the amount of cells is such that the concentration of cells in the scaffold is from about 800,000 cells/mm³ to about 1,500,000 cells/mm³.

15. The method of claim 12, wherein the skin precursor cells comprise epidermal and dermal precursor cells.

16. The method of claim 12, wherein the ratio of epidermal to dermal precursor cells is from about 1:5 to 1:10.

17. The method of claim 12, wherein the biocompatible scaffold is dried or lyophilized prior to admixing with the cells in serum-free medium.

18. The method of claim 12, wherein the scaffold and cells are admixed by passively contacting the cells with the scaffold at a temperature range from about 25° C. to about 37° C. for about 30 minutes to about 2 hours.

19. The method of claim 12, further comprising admixing an effective amount of an agent promoting cell differentiation or growth.

20. The method of claim 19, wherein the agent is one or more of Platelet Derived Growth Factor (PDGF), Vascular Endothelial Growth Factor (VEGF), Epithelial Growth Factor (EGF), TGF-β, Fibroblast Growth Factor (FGF), insulin, transferrin or retinoid.

21. The method of claim 12, further comprising admixing an effective amount of minoxidil, finasteride or an agent enhancing hair growth.

22. A method for generating pilosebaceous units in a physiological plane in a mammal in need thereof, comprising implanting the composition of claim 1, into the dermal layer of the mammal under a condition that favors implantation of the composition into the dermis of the mammal.

23. The method of claim 21, wherein the condition that favors implantation of the composition into the dermis of the mammal comprises applying suitable pressure to maintain contact between the composition and the muscle or subcutaneous fat of the mammal for at least 3 days.

24. The method of claim 22, wherein the dermal layer of the mammal was pretreated with an effective amount of an agent inhibiting the Bone Morphogenic Protein (BMP) signaling.

25. The method of claim 24, wherein the agent is one or more of dorsomorphin, noggin, chordin, gremlin, sclerostin or follistatin.

26. A method for preparing pilosebaceous units in a physiological plane, comprising admixing a number of skin precursor cells and a medium, wherein the concentration of dermal precursor cells present in the medium is from about 1×10⁷ cells per milliliter to about 1×10⁷ cells per milliliter.

27. The method of claim 26, wherein the skin precursor cells comprise epidermal and dermal precursor cells.

28. The method of claim 26, further comprising the step of overlaying an epithelial sheet.

29. A non-human animal model having the composition of claim 1 implanted into the dermis of the non-human animal.

30. A method for screening for an agent that modulates the generation of pilosebaceous units in a physiological plane in a mammal in need thereof, comprising adding an agent to be screened to a mammal having the composition of claim 1 implanted, and monitoring the growth of hair in the mammal, thereby screening for the agent.

31-32. (canceled)
